SEQUENCE LISTING (2 sequences)

SEQ ID NO: 1 - protein, 269 aa, Homo sapiens [sequence omitted]

SEQ ID NO: 2 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

(19) United States
(12) Patent Application Publication
(10) Pub. No.: US 2017/0313758 A1
(43) Pub. Date: Nov. 2, 2017

(54) Polypeptides for the Treatment of Angiogenesis or Lymphangiogenesis-Related Diseases

(71) Applicants: INSERM (Institut National de la Sante et de la Recherche Medicale), Paris (FR); Assistance Publique-Hopitaux de Paris (APHP), Paris (FR); Universite Paris Diderot - Paris 7, Paris (FR); Barcelona Supercomputing Center-Centro Nacional de Supercomputacion, Barcelona (ES); Centre National de la Recherche Scientifique (CNRS), Paris (FR); Universite Paris 13 - Paris Nord, Villetaneuse (FR); Universite Paris Descartes, Paris (FR)

(72) Inventors: Samia Mourah, Paris (FR); Bruno Villoutreix, Paris (FR); Farah Khayati, Boulogne-Billancourt (FR); Suzanne Menashi, Vincennes (FR); Celeste Lebbe, Paris (FR); Juan Fernandez Recio, Barcelona (ES); Fabien Calvo, Paris (FR)

(21) Appl. No.: 15/529,571

(22) PCT Filed: Nov. 24, 2015
(86) PCT No.: PCT/EP2015/077555

§ 371 Date: May 25, 2017

(30) Foreign Application Priority Data
Nov. 25, 2014 (EP) 14306880.7
Mar. 10, 2015 (EP) 15305360.8

(51) Int. Cl. C07K 14/705; C12N 15/115; C07K 16/28; A61K 38/00

(52) U.S. Cl. CPC C07K 14/70503; C07K 16/2803; C12N 15/115; C07K 2317/34; A61K 38/00; C12N 2310/16; C07K 2319/00

(57) ABSTRACT

The present invention relates to polypeptides for the treatment of angiogenesis or lymphangiogenesis-related diseases. In particular, the present invention relates to a a polypeptide which comprises or consists of a sequence of at least 5 consecutive amino acids in SEQ ID NO: 1 and which comprises at least one amino acid selected from the group consisting of Q182, R184, Q195, T199.

### FIELD OF THE INVENTION

The present invention relates to methods and pharmaceutical compositions for the treatment of angiogenesis or lymphangiogenesis-related diseases.

### BACKGROUND OF THE INVENTION

Angiogenesis is a key component of the tumor microenvironment, essential for tumor growth and invasion. Among the angiogenic regulators, vascular endothelial growth factor (VEGF) is known to be the major actor not only in endothelial cells but also in tumor cells, promoting survival, proliferation, apoptosis and migration [1].

VEGF exerts its angiogenic effects by binding to its main receptor (VEGFR-2) or KDR [1, 2]. Binding initiates receptor dimerization which subsequently activates the intracellular tyrosine kinase domains [2]. Active VEGFR-2 then initiates several downstream cell signaling pathways, including stress-activated protein kinase 2/p38 MAP kinase, phosphatidylinositol-f3 kinase, Focal Adhesion Kinase (FAK) and AKT, which culminate in endothelial cell migration, proliferation and vessel formation. The extracellular domain of VEGFR-2 consists of 7 Ig-homology domains. The first 3 domains were shown to mediate ligand binding whereas the membrane proximal domains are involved in ligand-induced receptor dimerization [3-5].

EMMPRIN/CD147, a membrane spanning glycoprotein particularly known as a regulator of matrix degrading proteinases such as MMPs and uPA, has been more recently shown to be implicated in angiogenesis via the regulation of VEGF expression [6-8]. The inventors described the concomitant regulation by EMMPRIN of VEGF receptor VEGFR-2 in both endothelial cells and tumor cells, in a mechanism mediated by HIF-2 alpha [9] thus increasing respectively angiogenesis and malignancy. It was also shown to have several other malignancy promoting functions including tumor cell invasion, survival and anchorage-independent growth [10]. Indeed, EMMPRIN has been greatly implicated in malignancy as it is highly expressed in most cancer tissues and its expression often correlates with tumor progression [11-14].

EMMPRIN belongs to the immunoglobulin (Ig) superfamily and is composed of two C2-like immunoglobulin extracellular domains, a transmembrane domain and a short cytoplasmic domain [15]. The extracellular region, which contains three conserved N-glycosylation sites that are variably glycosylated, has been implicated in EMMPRIN self association [16], while the first Ig domain within this region is required for counter-receptor activity involved in MMP induction [17]. The highly conserved transmembrane domain and the short cytoplasmic domain are thought to be implicated in interactions between EMMPRIN and other molecular partners within the membrane. In particular, EMMPRIN was shown to interact with integrins a3 01 and a6 01, enhancing the adhesion and spreading of the cell to the ECM [18] and to caveolin-1 in lipid rafts leading to a decrease in EMMPRIN cell surface self association [19].

The ability of EMMPRIN to associate with different proteins was suggested to determine different cellular functions, although the nature of such interactions and their involvement in signal transduction has not yet been determined. There is no disclosure in art of an inhibitor of EMMPRIN/VEGFR-2 interaction, nor their use in the inhibition of angiogenesis, the inhibition of lymphangiogenesis, or in the treatment of angiogenesis or lymphoangiogenesis-related diseases.

### SUMMARY OF THE INVENTION

The present invention relates to polypeptides for the treatment of angiogenesis or lymphangiogenesis-related diseases. In particular, the present invention is defined by the claims.

### DETAILED DESCRIPTION OF THE INVENTION

In the present invention, the inventors investigated the role of EMMPRIN and VEGF-2 in tumor angiogenesis. The inventors provides evidence that EMMPRIN is a new coreceptor for the VEGFR-2 tyrosine kinase receptor in both endothelial and tumor cells, as it directly interacts with it and regulates its activation by its VEGF ligand, signalling and functional consequences both in vitro and in vivo. The inventors identified a molecular binding site in the extracellular domain of EMMPRIN located close to the cell membrane and containing the amino acids 195/199 using computational docking analyses and mutagenesis. EMMPRIN is known to be overexpressed in cancer and hence is able to further potentiate VEGFR-2 activation, demonstrating that a combinatory therapy of an antiangiogenic drug together with an inhibitor of EMMPRINNEGFR-2 interaction have a greater impact on inhibiting angiogenesis and malignancy.

### Polypeptides of the Invention

The present invention relates to an isolated, synthetic or recombinant polypeptides which is an inhibitor of EMMPRIN/VEGFR-2 interaction.

As used herein the term “EMMPRIN” has its general meaning in the art and refers to CD147, a membrane spanning glycoprotein particularly known as a regulator of matrix degrading proteinases such as MMPs and uPA, has been more recently shown to be implicated in angiogenesis via the regulation of VEGF expression [6-8]. EMMPRIN belongs to the immunoglobulin (Ig) superfamily and is composed of two C2-like immunoglobulin extracellular domains, a transmembrane domain and a short cytoplasmic domain [15]. An exemplary human polypeptide sequence of EMMPRIN is SEQ ID NO:1 (as shown in FIG. 17).

SEQ ID NO: 1
MAAALFVLLGFALLGTHGASGAAGTVFTTVEDLGSKILLTCSLNDSATEV
TGHRWLKGGVVLKEDALPGQKTEFKVDSDDQWGEYSCVFPPEPMGTANIQ
LHGPPRVKAVKSSEHINEGETAMLVCKSESVPPVTDWAWYKITDSEDKAL
MNGSESRFFVSSSQGRSELHIENLNMEADPGQYRCNGTSSKGSDQAIITL
RVCSHLAALWPFLGIVAEVLVLVTIIFIYEKRRKPEDVLDDDDAGSAPLK
SSGQHQNDKGKNVRQRNSS

As used herein the term “VEGFR-2” has its general meaning in the art and refers to the subtype 2 of VEGF receptor or vascular endothelial growth factor receptor. An exemplary human polypeptide sequence of VEGFR-2 is SEQ ID NO:2

SEQ ID NO: 2
MQSKVLLAVALWLCVETRAASVGLPSVSLDLPRLSIQKDILTIKANTTLQ
ITCRGQRDLDWLWPNNQSGSEQRVEVTECSDGLFCKTLTIPKVIGNDTGA
YKCFYRETDLASVIYVYVQDYRSPFIASVSDQHGVVYITENKNKTVVIPC
LGSISNLNVSLCARYPEKRFVPDGNRISWDSKKGFTIPSYMISYAGMVFC
EAKINDESYQSIMYIVVVVGYRIYDVVLSPSHGIELSVGEKLVLNCTART
ELNVGIDFNWEYPSSKHQHKKLVNRDLKTQSGSEMKKFLSTLTIDGVTRS
DQGLYTCAASSGLMTKKNSTFVRVHEKPFVAFGSGMESLVEATVGERVRI
PAKYLGYPPPEIKWYKNGIPLESNHTIKAGHVLTIMEVSERDTGNYTVIL
TNPISKEKQSHVVSLVVYVPPQIGEKSLISPVDSYQYGTTQTLTCTVYAI
PPPHHIHWYWQLEEECANEPSQAVSVTNPYPCEEWRSVEDFQGGNKIEVN
KNQFALIEGKNKTVSTLVIQAANVSALYKCEAVNKVGRGERVISFHVTRG
PEITLQPDMQPTEQESVSLWCTADRSTFENLTWYKLGPQPLPIHVGELPT
PVCKNLDTLWKLNATMFSNSTNDILIMELKNASLQDQGDYVCLAQDRKTK
KRHCVVRQLTVLERVAPTITGNLENQTTSIGESIEVSCTASGNPPPQIMW
FKDNETLVEDSGIVLKDGNRNLTIRRVRKEDEGLYTCQACSVLGCAKVEA
FFIIEGAQEKTNLEIIILVGTAVIAMFFWLLLVIILRTVKRANGGELKTG
YLSIVMDPDELPLDEHCERLPYDASKWEFPRDRLKLGKPLGRGAFGQVIE
ADAFGIDKTATCRTVAVKMLKEGATHSEHRALMSELKILIHIGHHLNVVN
LLGACTKPGGPLMVIVEFCKFGNLSTYLRSKRNEFVPYKTKGARFRQGKD
YVGAIPVDLKRRLDSITSSQSSASSGFVEEKSLSDVEEEEAPEDLYKDFL
TLEHLICYSFQVAKGMEFLASRKCIHRDLAARNILLSEKNVVKICDFGLA
RDIYKDPDYVRKGDARLPLKWMAPETIFDRVYTIQSDVWSFGVLLWEIFS
LGASPYPGVKIDEEFCRRLKEGTRMRAPDYTTPEMYQTMLDCWHGEPSQR
PTFSELVEHLGNLLQANAQQDGKDYIVLPISETLSMEEDSGLSLPTSPVS
CMEEEEVCDPKFHYDNTAGISQYLQNSKRKSRPVSVKTFEDIPLEEPEVK
VIPDDNQTDSGMVLASEELKTLEDRTKLSPSEGGMVPSKSRESVASEGSN
QTSGYQSGYHSDDTDTTVYSSEEAELLKLIEIGVQTGSTAQILQPDSGTT
LSSPPV

The “EMMPRIN/VEGFR-2 molecular binding site” refers to binding site between the extracellular domain of EMMPRIN which contains the amino acids Q182/R184/Q195/T199 of SEQ ID NO:1 and domains D6 and D7 of EMMPRIN. In particular, the binding site of EMMPRIN is located between the amino acid residue at position 130 to the amino acid at position 210 in SEQ ID NO:1

As used herein, the term “inhibitor of EMMPRIN/VEGFR-2 interaction” refers to any compound that is able to inhibit the interaction between EMMPRIN and VEGFR2 at the EMMPRIN/VEGFR-2 molecular binding site. In some embodiments, the compound bind to EMMPRIN or binds to VEGFR2. In some embodiments, the compound bind to the region ranging from the amino acid residue at position 130 to the amino acid at position 210 in SEQ ID NO:1.

In some embodiments, the polypeptide of the present invention comprises or consists of a sequence of at least 5 consecutive amino acids in SEQ ID NO:1 and which comprises at least one amino acid selected from the group consisting of Q182, R184, Q195, T199.

In some embodiments, the polypeptide of the present invention comprises or consists of a sequence of at least 5 consecutive amino acids in SEQ ID NO:1 and which comprises at least two amino acids selected from the group consisting of Q182, R184, Q195, T199.

In some embodiments, the polypeptide of the present invention comprises or consists of a sequence of at least 5 consecutive amino acids in SEQ ID NO:1 and which comprises at least three amino acids selected from Q182, R184, Q195, T199.

In some embodiments, the polypeptide of the present invention comprises or consists of a sequence of at least 5 consecutive amino acids in SEQ ID NO:1 and which comprises all of the amino acids Q182/R184/Q195/T199.

In some embodiments, the polypeptide of the present invention comprises or consists of a sequence of at least 5 consecutive amino acids in the region ranging from the residue at position 130 to the amino acid residue at position 210.

In some embodiments, the polypeptide of the present invention comprises or consists of a sequence of at least 5 consecutive amino acids in the region ranging from the residue at position 190 to the amino acid residue at position 202.

In some embodiments, the polypeptide of the invention comprises 5; 6; 7; 8; 9; 10; 11; 12; 13; 14; 15; 16; 17; 18; 19; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; or 50 consecutive amino acids in SEQ ID NO:1.

In some embodiments, the polypeptide of the present invention comprises or consists of the sequence having at least 70% of identity with the sequence which ranges from the amino acid residue at position 190 to the amino acid residue at position 202 in SEQ ID NO:1 (sequence P1 in FIG. 17).

In some embodiments, the polypeptide of the present invention comprises or consists of the sequence having at least 70% of identity with the sequence which ranges from the amino acid residue at position 179 to the amino acid residue at position 192 in SEQ ID NO:1.

In some embodiments the polypeptide of the present invention comprises or consists of the sequence having at least 70% of identity with the sequence which ranges from the amino acid residue at position 181 to the amino acid residue at position 192 in SEQ ID NO:1.

According to the invention a first amino acid sequence having at least 70% of identity with a second amino acid sequence means that the first sequence has 70; 71; 72; 73; 74; 75; 76; 77; 78; 79; 80; 81; 82; 83; 84; 85; 86; 87; 88; 89; 90; 91; 92; 93; 94; 95; 96; 97; 98; or 99, or 100% of identity with the second amino acid sequence. Amino acid sequence identity is preferably determined using a suitable sequence alignment algorithm and default parameters, such as BLAST P (Karlin and Altschul, 1990).

In some embodiments, the polypeptide of the present invention does not consist of the amino acid sequence SEQ ID NO:1 and does not consist of the amino acid sequence ranging from the histidine residue at position 170 to the arginine residue at position 184 in SEQ ID NO:1.

A further aspect of the present invention relates to a fusion protein comprising a polypeptide of to the invention that is fused to at least one heterologous polypeptide.

The term “fusion protein” refers to the polypeptide of the invention that is fused directly or via a spacer to at least one heterologous polypeptide.

According to the invention, the fusion protein comprises the polypeptide of the invention that is fused either directly or via a spacer at its C-terminal end to the N-terminal end of the heterologous polypeptide, or at its N-terminal end to the C-terminal end of the heterologous polypeptide.

As used herein, the term “directly” means that the (first or last) amino acid at the terminal end (N or C-terminal end) of the polypeptide is fused to the (first or last) amino acid at the terminal end (N or C-terminal end) of the heterologous polypeptide.

In other words, in this embodiment, the last amino acid of the C-terminal end of said polypeptide is directly linked by a covalent bond to the first amino acid of the N-terminal end of said heterologous polypeptide, or the first amino acid of the N-terminal end of said polypeptide is directly linked by a covalent bond to the last amino acid of the C-terminal end of said heterologous polypeptide.

As used herein, the term “spacer” refers to a sequence of at least one amino acid that links the polypeptide of the invention to the heterologous polypeptide. Such a spacer may be useful to prevent steric hindrances. Typically a spacer comprises 3; 4; 5; 6; 7; 8; 9; 10; 11; 12; 13; 14; 15; 16; 17; 18; 19; or 20 amino acids.

In some embodiment, the heterologous polypeptide is a vascular or tumor targeting agent. Said vascular and/or tumor targeting agent include but are not limited to antibodies directed against the EDB domain of fibronectin, antibodies or agents binding Vascular endothelial growth factor receptor 2, antibodies or molecules binding fibroblast growth factor receptor-1, antibodies or agents that interact with CD31, antibodies or agents interacting with tumor lymphatic endothelium (Podoplanin, Lyve-1), or antibodies or agents binding to αVβ3 integrin such as RGD peptides, or antibodies or agents interacting with tumor membrane-bound and intracellular targets. Strategies for vascular targeting in tumors have been reviewed for instance by Brekken et al. (Int. J. Cancer. 2002;100 (2): 123-130).

In some embodiments, the heterologous polypeptide is a cell-penetrating peptide which is typically, a Transactivator of Transcription (TAT) cell penetrating sequence, a cell permeable peptide or a membranous penetrating sequence. The term “cell-penetrating peptides” are well known in the art and refers to cell permeable sequence or membranous penetrating sequence such as penetratin, TAT mitochondrial penetrating sequence and compounds (Bechara and Sagan, 2013; Jones and Sayers, 2012; Khafagy el and Morishita, 2012; Malhi and Murthy, 2012). In some embodiments, the heterologous polypeptide is an internalization sequence derived either from the homeodomain of Drosophila Antennapedia/Penetratin (Antp) protein or a Transactivator of Transcription (TAT) cell penetrating sequence.

The polypeptides or fusion proteins of the invention are produced by any technique known per se in the art, such as, without limitation, any chemical, biological, genetic or enzymatic technique, either alone or in combination. For instance, knowing the amino acid sequence of the desired sequence, one skilled in the art can readily produce said polypeptides or fusion proteins, by standard techniques for production of amino acid sequences. For instance, they can be synthesized using well-known solid phase method, preferably using a commercially available peptide synthesis apparatus (such as that made by Applied Biosystems, Foster City, Calif.) and following the manufacturer's instructions. Alternatively, the polypeptides or fusion proteins of the invention can be synthesized by recombinant DNA techniques as is now well-known in the art. For example, these fragments can be obtained as DNA expression products after incorporation of DNA sequences encoding the desired (poly)peptide into expression vectors and introduction of such vectors into suitable eukaryotic or prokaryotic hosts that will express the desired polypeptide, from which they can be later isolated using well-known techniques.

Polypeptides or fusion proteins of the invention can be used in an isolated (e.g., purified) form or contained in a vector, such as a membrane or lipid vesicle (e.g. a liposome).

In specific embodiments, it is contemplated that polypeptides or fusion proteins according to the invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain. For example, Pegylation is a well-established and validated approach for the modification of a range of polypeptides (Chapman, 2002). The benefits include among others: (a) markedly improved circulating half-lives in vivo due to either evasion of renal clearance as a result of the polymer increasing the apparent size of the molecule to above the glomerular filtration limit, and/or through evasion of cellular clearance mechanisms; (b) reduced antigenicity and immunogenicity of the molecule to which PEG is attached; (c) improved pharmacokinetics; (d) enhanced proteolytic resistance of the conjugated protein (Cunningham-Rundles et. al., 1992); and (e) improved thermal and mechanical stability of the PEGylated polypeptide. Therefore, advantageously, the polypeptides of the invention may be covalently linked with one or more polyethylene glycol (PEG) group(s). One skilled in the art can select a suitable molecular mass for PEG, based on how the pegylated polypeptide will be used therapeutically by considering different factors including desired dosage, circulation time, resistance to proteolysis, immunogenicity, etc. In some embodiments, additional sites for PEGylation can be introduced by site-directed mutagenesis by introducing one or more lysine residues. For instance, one or more arginine residues may be mutated to a lysine residue. In some embodiments, additional PEGylation sites are chemically introduced by modifying amino acids on polypeptides of the invention. In some embodiments, PEGs are conjugated to the polypeptides or fusion proteins through a linker. Suitable linkers are well known to the skilled person.

### Nucleic Acids, Vectors and Recombinant Host Cells

A further object of the present invention relates to a nucleic acid sequence encoding for a polypeptide or a fusion protein according to the invention.

As used herein, a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

These nucleic acid sequences can be obtained by conventional methods well known to those skilled in the art. Typically, said nucleic acid is a DNA or RNA molecule, which may be included in a suitable vector, such as a plasmid, cosmid, episome, artificial chromosome, phage or viral vector.

So, a further object of the present invention relates to a vector and an expression cassette in which a nucleic acid molecule encoding for a polypeptide or a fusion protein of the invention is associated with suitable elements for controlling transcription (in particular promoter, enhancer and, optionally, terminator) and, optionally translation, and also the recombinant vectors into which a nucleic acid molecule in accordance with the invention is inserted. These recombinant vectors may, for example, be cloning vectors, or expression vectors.

As used herein, the terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence.

Any expression vector for animal cell can be used. Examples of suitable vectors include pAGE107 (Miyaji et al., 1990), pAGE103 (Mizukami and Itoh, 1987), pHSG274 (Brady et al., 1984), pKCR (O'Hare et al., 1981), pSG1 beta d2-4 (Miyaji et al., 1990) and the like. Other examples of plasmids include replicating plasmids comprising an origin of replication, or integrative plasmids, such as for instance pUC, pcDNA, pBR, and the like. Other examples of viral vectors include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. No. 5,882,877, U.S. Pat. No. 6,013,516, U.S. Pat. No. 4,861,719, U.S. Pat. No. 5,278,056 and WO 94/19478. Examples of promoters and enhancers used in the expression vector for animal cell include early promoter and enhancer of SV40 (Mizukami and Itoh, 1987), LTR promoter and enhancer of Moloney mouse leukemia virus (Kuwana et al., 1987), promoter (Mason et al., 1985) and enhancer (Gillies et al., 1983) of immunoglobulin H chain and the like.

A further aspect of the invention relates to a host cell comprising a nucleic acid molecule encoding for a polypeptide or a fusion protein according to the invention or a vector according to the invention. In particular, a subject of the present invention is a prokaryotic or eukaryotic host cell genetically transformed with at least one nucleic acid molecule or vector according to the invention.

The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed”.

In some embodiments, for expressing and producing polypeptides or fusion proteins of the invention, prokaryotic cells, in particular E. coli cells, will be chosen. Actually, according to the invention, it is not mandatory to produce the polypeptide or the fusion protein of the invention in a eukaryotic context that will favour post-translational modifications (e.g.

glycosylation). Furthermore, prokaryotic cells have the advantages to produce protein in large amounts. If a eukaryotic context is needed, yeasts (e.g. saccharomyces strains) may be particularly suitable since they allow production of large amounts of proteins. Otherwise, typical eukaryotic cell lines such as CHO, BHK-21, COS-7, C127, PER.C6, YB2/0 or HEK293 could be used, for their ability to process to the right post-translational modifications of the fusion protein of the invention.

The construction of expression vectors in accordance with the invention, and the transformation of the host cells can be carried out using conventional molecular biology techniques. The polypeptide or the fusion protein of the invention, can, for example, be obtained by culturing genetically transformed cells in accordance with the invention and recovering the polypeptide or the fusion protein expressed by said cell, from the culture. They may then, if necessary, be purified by conventional procedures, known in themselves to those skilled in the art, for example by fractional precipitation, in particular ammonium sulfate precipitation, electrophoresis, gel filtration, affinity chromatography, etc. In particular, conventional methods for preparing and purifying recombinant proteins may be used for producing the proteins in accordance with the invention.

A further aspect of the invention relates to a method for producing a polypeptide or a fusion protein of the invention comprising the step consisting of: (i) culturing a transformed host cell according to the invention under conditions suitable to allow expression of said polypeptide or fusion protein; and (ii) recovering the expressed polypeptide or fusion protein.

### Aptamers and Antibodies:

The present invention also related to an antibody or an aptamer which specifically binds to a polypeptide of the present invention.

In some embodiments, the aptamer or antibody of the present invention specifically bind to the polypeptide which comprises or consists of a sequence having at least 70% of identity with the sequence which ranges from the amino acid residue at position 190 to the amino acid residue at position 202 in SEQ ID NO:1 (sequence P1 in FIG. 17). In some embodiments, the the aptamer or antibody of the present invention specifically bind to the polypeptide having the sequence which ranges from the amino acid residue at position 190 to the amino acid residue at position 202 in SEQ ID NO:1 (sequence P1 in FIG. 17).

The term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/11161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments.

In natural antibodies, two heavy chains are linked to each other by disulfide bonds and each heavy chain is linked to a light chain by a disulfide bond. There are two types of light chain, lambda (1) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, includes six CDRs, comprising the CDR set from each of a heavy and a light chain V region. Framework Regions (FRs) refer to amino acid sequences interposed between CDRs.

The term “Fab” denotes an antibody fragment having a molecular weight of about 50,000 and antigen binding activity, in which about a half of the N-terminal side of H chain and the entire L chain, among fragments obtained by treating IgG with a protease, papaine, are bound together through a disulfide bond.

The term “F(ab′)2” refers to an antibody fragment having a molecular weight of about 100,000 and antigen binding activity, which is slightly larger than the Fab bound via a disulfide bond of the hinge region, among fragments obtained by treating IgG with a protease, pepsin.

The term “Fab′” refers to an antibody fragment having a molecular weight of about 50,000 and antigen binding activity, which is obtained by cutting a disulfide bond of the hinge region of the F(ab′)2.

A single chain Fv (“scFv”) polypeptide is a covalently linked VH::VL heterodimer which is usually expressed from a gene fusion including VH and VL encoding genes linked by a peptide-encoding linker. “dsFv” is a VH::VL heterodimer stabilised by a disulfide bond. Divalent and multivalent antibody fragments can form either spontaneously by association of monovalent scFvs, or can be generated by coupling monovalent scFvs by a peptide linker, such as divalent sc(Fv)2.

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites.

Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with the appropriate antigenic forms (i.e. polypeptides of the present invention). The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the recombinant polypeptide of the invention may be provided by expression with recombinant cell lines. Recombinant forms of the polypeptides may be provided using any previously described method. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods. Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc' regions to produce a functional antibody.

In some embodiments, the antibody is a humanized antibody. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In some embodiments, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., J. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans. In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4.

Aptamers are a class of molecule that represents an alternative to antibodies in term of molecular recognition. Aptamers are oligonucleotide sequences with the capacity to recognize virtually any class of target molecules with high affinity and specificity. Such ligands may be isolated through Systematic Evolution of Ligands by EXponential enrichment (SELEX) of a random sequence library. The random sequence library is obtainable by combinatorial chemical synthesis of DNA. In this library, each member is a linear oligomer, eventually chemically modified, of a unique sequence. Peptide aptamers consists of a conformationally constrained antibody variable region displayed by a platform protein, such as E. coli

Thioredoxin A that are selected from combinatorial libraries by two hybrid methods (Colas et al., 1996).

### Therapeutic Methods and Uses

According to the invention, the polypeptides, nucleic acids, aptamers and antibodies of the present invention are particularly suitable of inhibiting the interaction between EMMPRIN and VEGFR2. The polypeptides, nucleic acids, aptamers and antibodies of the present invention are thus particularly suitable for inhibiting the effects mediated by VEGFR2 see EXAMPLE) which include angiogenesis and lymphoangiogenesis.

A further aspect of the invention relates to an agent selected from the group consisting of polypeptides, nucleic acids, aptamers and antibodies of the present invention for use in the treatment of angiogenesis-related diseases or lymphoangiogenesis-related diseases in a subject in need thereof.

As used herein, the term “subject” denotes a mammal. Typically, a subject according to the invention refers to any subject (preferably human) afflicted with or susceptible to be afflicted with angiogenesis-related diseases or lymphoangiogenesis-related diseases. Typically a subject according to the invention is a subject afflicted or susceptible to be afflicted with a cancer.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “angiogenesis-related diseases” has its general meaning in the art and refers to diseases associated with or supported by pathological angiogenesis (i.e., inappropriate, excessive or undesired formation of blood vessels), which may be induced by various angiogenic factors. The term “angiogenesis-related diseases” also relates to angiogenic diseases associated with abnormal neovascularisation. Angiogenesis-related diseases include but are not limited to cancer, tumor angiogenesis, primary and metastatic solid tumors, including carcinomas of breast, colon, rectum, lung, oropharynx, hypopharynx, esophagus, stomach, pancreas, liver, gallbladder and bile ducts, small intestine, kidney, bladder, urothelium, female genital tract, (including cervix, uterus, and ovaries as well as choriocarcinoma and gestational trophoblastic disease), male genital tract (including prostate, seminal vesicles, testes and germ cell tumors), endocrine glands (including the thyroid, adrenal, and pituitary glands), and skin, as well as hemangiomas, melanomas, sarcomas (including those arising from bone and soft tissues as well as Kaposi's sarcoma) and tumors of the brain, nerves, eyes, such as astrocytomas, gliomas, glioblastomas, retinoblastomas, neuromas, neuroblastomas, Schwannomas, and meningiomas. Angiogenesis-related diseases also relate to tumors arising from hematopoietic malignancies such as leukemias as well both Hodgkin's and non-Hodgkin's lymphomas. Angiogenesis-related diseases also relate to various ocular diseases such as diabetic retinopathy, retinopathy of prematurity, corneal graft rejection, retrolental fibroplasia, neovascular glaucoma, rubeosis, retinal neovascularization due to macular degeneration, hypoxia, angiogenesis in the eye associated with infection or surgical intervention, and other abnormal neovascularization conditions of the eye. Angiogenesis-related diseases also relate to rheumatoid, immune and degenerative arthritis. Angiogenesis-related diseases also relate to skin diseases such as psoriasis; blood vessel diseases such as hemagiomas, and capillary proliferation within atherosclerotic plaques; Osler-Webber Syndrome; myocardial angiogenesis; plaque neovascularization; telangiectasia; hemophiliacjoints'; angiofibroma; and wound granulation. Angiogenesis-related diseases also relate to diseases characterized by excessive or abnormal stimulation of endothelial cells, including but not limited to intestinal adhesions, Crohn's disease, atherosclerosis, scleroderma, and hypertrophic scars, i.e. keloids., diseases that have angiogenesis as a pathologic consequence such as cat scratch disease (Rochele ninalia quintosa) and ulcers (Helicobacter pylori).

As used herein, the term “lymphoangiogenesis-related diseases” has its general meaning in the art and refers to pathological conditions or disorders associated with lymphangiogenesis (i.e. abnormal lymphangiogenesis). The term “lymphoangiogenesis-related diseases” includes, but is not limited to cancer, eye diseases (such as corneal graft rejection, age-related macular degeneration and diabetic retinopathy) and inflammatory diseases (such as rheumatoid arthritis and psoriasis). As used herein, the term “lymphangiogenesis” refers to growth of new lymphatic vessels.

In some embodiments, the agent selected from the group consisting of polypeptides, nucleic acids, aptamers and antibodies of the present invention is particularly suitable for the treatment of cancers that are resistant to tyrosine kinase inhibitors (TKI). The term “tyrosine kinase inhibitor” refers to any of a variety of therapeutic agents or drugs that act as selective or non-selective inhibitors of receptor and/or non-receptor tyrosine kinases. Tyrosine kinase inhibitors and related compounds are well known in the art and described in U.S. Patent Publication 2007/0254295, which is incorporated by reference herein in its entirety. It will be appreciated by one of skill in the art that a compound related to a tyrosine kinase inhibitor will recapitulate the effect of the tyrosine kinase inhibitor, e.g., the related compound will act on a different member of the tyrosine kinase signaling pathway to produce the same effect as would a tyrosine kinase inhibitor of that tyrosine kinase. Examples of tyrosine kinase inhibitors and related compounds suitable for use in methods of embodiments of the present invention include, but are not limited to, dasatinib (BMS-354825), PP2, BEZ235, saracatinib, gefitinib (Iressa), sunitinib (Sutent; SU11248), erlotinib (Tarceva; OSI-1774), lapatinib (GW572016; GW2016), canertinib (CI 1033), semaxinib (SU5416), vatalanib (PTK787/ZK222584), sorafenib (BAY 43-9006), imatinib (Gleevec; STI571), leflunomide (SU101), vandetanib (Zactima; ZD6474), MK-2206 (8-[4-aminocyclobutyl)phenyl]-9-phenyl-1,2,4-triazolo[3,4-f][1,6]naphthyridin-3 (2H)-one hydrochloride) derivatives thereof, analogs thereof, and combinations thereof. Additional tyrosine kinase inhibitors and related compounds suitable for use in the present invention are described in, for example, U.S Patent Publication 2007/0254295, U.S. Pat. Nos. 5,618,829, 5,639,757, 5,728,868, 5,804,396, 6,100,254, 6,127,374, 6,245,759, 6,306,874, 6,313,138, 6,316,444, 6,329,380, 6,344,459, 6,420,382, 6,479,512, 6,498,165, 6,544,988, 6,562,818, 6,586,423, 6,586,424, 6,740,665, 6,794,393, 6,875,767, 6,927,293, and 6,958,340, all of which are incorporated by reference herein in their entirety. In certain embodiments, the tyrosine kinase inhibitor is a small molecule kinase inhibitor that has been orally administered and that has been the subject of at least one Phase I clinical trial, more preferably at least one Phase II clinical, even more preferably at least one Phase III clinical trial, and most preferably approved by the FDA for at least one hematological or oncological indication. Examples of such inhibitors include, but are not limited to, Gefitinib, Erlotinib, Lapatinib, Canertinib, BMS-599626 (AC-480), Neratinib, KRN-633, CEP-11981, Imatinib, Nilotinib, Dasatinib, AZM-475271, CP-724714, TAK-165, Sunitinib, Vatalanib, CP-547632, Vandetanib, Bosutinib, Lestaurtinib, Tandutinib, Midostaurin, Enzastaurin, AEE-788, Pazopanib, Axitinib, Motasenib, OSI-930, Cediranib, KRN-951, Dovitinib, Seliciclib, SNS-032, PD-0332991, MKC-I (Ro-317453; R-440), Sorafenib, ABT-869, Brivanib (BMS-582664), SU-14813, Telatinib, SU-6668, (TSU-68), L-21649, MLN-8054, AEW-541, and PD-0325901. Typically, the TKI of the present invention is suitable for inhibiting VEGFR2. In some embodiments, the agent selected from the group consisting of polypeptides, nucleic acids, aptamers and antibodies of the present invention is particularly suitable for the treatment of cancers that are resistant to sutent (e.g. renal cell carcinoma resistant to sutent).

Typically the agent of the present invention as described above is administered to the subject in a therapeutically effective amount.

By a “therapeutically effective amount” of the agent of the present invention as above described is meant a sufficient amount of the compound. It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typicially, the compositions contain 0.01, 0.05, 0.1, 0.5, 1.0, 2.5, 5.0, 10.0, 15.0, 25.0, 50.0, 100, 250 and 500 mg of the agent of the present invention for the symptomatic adjustment of the dosage to the subject to be treated. A medicament typically contains from about 0.01 mg to about 500 mg of the agent of the present invention, preferably from 1 mg to about 100 mg of the agent of the present invention. An effective amount of the drug is ordinarily supplied at a dosage level from 0.0002 mg/kg to about 20 mg/kg of body weight per day, especially from about 0.001 mg/kg to 7 mg/kg of body weight per day.

According to the invention, the agent of the present invention is administered to the subject in the form of a pharmaceutical composition. Typically, the agent of the present invention may be combined with pharmaceutically acceptable excipients, and optionally sustained-release matrices, such as biodegradable polymers, to form therapeutic compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The agent of the present invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized agent of the present inventions into a sterile vehicle which contains the basic dispersion medium and the required other ingredients from those enumerated above. In the case of sterile powders for the preparation of sterile injectable solutions, the typical methods of preparation are vacuum-drying and freeze-drying techniques which yield a powder of the agent of the present invention plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

In some embodiments, the agent of the present invention is administered sequentially or concomitantly with one or more therapeutic active agent such as chemotherapeutic, radiotherapeutic, anti-angiogenic (including targeted therapy . . . ) or anti-lymphangiogenic agents.

In some embodiments, the agent of the present invention is administered with a chemotherapeutic agent. The term “chemotherapeutic agent” refers to chemical compounds that are effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33:183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmo fur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; amino levulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; capecitabine; and phannaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazo les, 4-hydroxytamoxifen, trioxifene, keoxifene, LY117018, onapristone, and toremifene (Fareston); and anti-androgens such as flutamide, nilutamide, bicalutamide, leuprolide, and goserelin; and phannaceutically acceptable salts, acids or derivatives of any of the above.

In some embodiments, the agent of the present invention is administered with a targeted cancer therapy. Targeted cancer therapies are drugs or other substances that block the growth and spread of cancer by interfering with specific molecules (“molecular targets”) that are involved in the growth, progression, and spread of cancer. Targeted cancer therapies are sometimes called “molecularly targeted drugs,” “molecularly targeted therapies,” “precision medicines,” or similar names. In some embodiments, the targeted therapy consists of administering the subject with a tyrosine kinase inhibitor as defined above.

In some embodiments, agent of the present invention is administered with an immunotherapeutic agent. The term “immunotherapeutic agent,” as used herein, refers to a compound, composition or treatment that indirectly or directly enhances, stimulates or increases the body's immune response against cancer cells and/or that decreases the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, cytokines, cancer vaccines, monoclonal antibodies and non-cytokine adjuvants. Alternatively the immunotherapeutic treatment may consist of administering the subject with an amount of immune cells (T cells, NK, cells, dendritic cells, B cells . . . ). Immunotherapeutic agents can be non-specific, i.e. boost the immune system generally so that the human body becomes more effective in fighting the growth and/or spread of cancer cells, or they can be specific, i.e. targeted to the cancer cells themselves immunotherapy regimens may combine the use of non-specific and specific immunotherapeutic agents. Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants. A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors. Interferons (IFNs) contemplated by the present invention include the common types of IFNs, IFN-alpha (IFN-α), IFN-beta (IFN-β) and IFN-gamma (IFN-γ). IFNs can act directly on cancer cells, for example, by slowing their growth, promoting their development into cells with more normal behaviour and/or increasing their production of antigens thus making the cancer cells easier for the immune system to recognise and destroy. IFNs can also act indirectly on cancer cells, for example, by slowing down angiogenesis, boosting the immune system and/or stimulating natural killer (NK) cells, T cells and macrophages. Recombinant IFN-alpha is available commercially as Roferon (Roche Pharmaceuticals) and Intron A (Schering Corporation). Interleukins contemplated by the present invention include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention. Colony-stimulating factors (CSFs) contemplated by the present invention include granulocyte colony stimulating factor (G-CSF or filgrastim), granulocyte-macrophage colony stimulating factor (GM-CSF or sargramostim) and erythropoietin (epoetin alfa, darbepoietin). Treatment with one or more growth factors can help to stimulate the generation of new blood cells in subjects undergoing traditional chemotherapy. Accordingly, treatment with CSFs can be helpful in decreasing the side effects associated with chemotherapy and can allow for higher doses of chemotherapeutic agents to be used. Various-recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Arnesp (erytropoietin). In addition to having specific or non-specific targets, immunotherapeutic agents can be active, i.e. stimulate the body's own immune response, or they can be passive, i.e. comprise immune system components that were generated external to the body. Passive specific immunotherapy typically involves the use of one or more monoclonal antibodies that are specific for a particular antigen found on the surface of a cancer cell or that are specific for a particular cell growth factor. Monoclonal antibodies may be used in the treatment of cancer in a number of ways, for example, to enhance a subject's immune response to a specific type of cancer, to interfere with the growth of cancer cells by targeting specific cell growth factors, such as those involved in angiogenesis, or by enhancing the delivery of other anticancer agents to cancer cells when linked or conjugated to agents such as chemotherapeutic agents, radioactive particles or toxins. Monoclonal antibodies currently used as cancer immunotherapeutic agents that are suitable for inclusion in the combinations of the present invention include, but are not limited to, rituximab (Rituxan®), trastuzumab (Herceptin®), ibritumomab tiuxetan (Zevalin®), tositumomab (Bexxar®), cetuximab (C-225, Erbitux®), bevacizumab (Avastin®), gemtuzumab ozogamicin (Mylotarg®), alemtuzumab (Campath®), and BL22. Other examples include anti-CTLA4 antibodies (e.g. Ipilimumab), anti-PD1 antibodies, anti-PDLL antibodies, anti-TIMP3 antibodies, anti-LAG3 antibodies, anti-B7H3 antibodies, anti-B7H4 antibodies or anti-B7H6 antibodies. In some embodiments, antibodies include B cell depleting antibodies. Typical B cell depleting antibodies include but are not limited to anti-CD20 monoclonal antibodies [e.g. Rituximab (Roche), Ibritumomab tiuxetan (Bayer Schering), Tositumomab (GlaxoSmithKline), AME-133v (Applied Molecular Evolution), Ocrelizumab (Roche), Ofatumumab (HuMax-CD20, Gemnab), TRU-015 (Trubion) and IMMU-106 (Immunomedics)], an anti-CD22 antibody [e.g. Epratuzumab, Leonard et al., Clinical Cancer Research (Z004) 10: 53Z7-5334], anti-CD79a antibodies, anti-CD27 antibodies, or anti-CD19 antibodies (e.g. U.S. Pat. No. 7,109,304), anti-BAFF-R antibodies (e.g. Belimumab, GlaxoSmithKline), anti-APRIL antibodies (e.g. anti-human APRIL antibody, ProSci inc.), and anti-IL-6 antibodies [e.g. previously described by De Benedetti et al., J Immunol (2001) 166: 4334-4340 and by Suzuki et al., Europ J of Immunol (1992) 22 (8) 1989-1993, fully incorporated herein by reference]. The immunotherapeutic treatment may consist of allografting, in particular, allograft with hematopoietic stem cell HSC. The immunotherapeutic treatment may also consist in an adoptive immunotherapy as described by Nicholas P. Restifo, Mark E. Dudley and Steven A. Rosenberg “Adoptive immunotherapy for cancer: harnessing the T cell response, Nature Reviews Immunology, Volume 12, April 2012). In adoptive immunotherapy, the subject's circulating lymphocytes, NK cells, are isolated amplified in vitro and readministered to the subject. The activated lymphocytes or NK cells are most preferably be the subject's own cells that were earlier isolated from a blood or tumor sample and activated (or “expanded”) in vitro.

In some embodiments, the agent of the present invention is administered with a radiotherapeutic agent. The term “radiotherapeutic agent” as used herein, is intended to refer to any radiotherapeutic agent known to one of skill in the art to be effective to treat or ameliorate cancer, without limitation. For instance, the radiotherapeutic agent can be an agent such as those administered in brachytherapy or radionuclide therapy. Such methods can optionally further comprise the administration of one or more additional cancer therapies, such as, but not limited to, chemotherapies, and/or another radiotherapy.

In one embodiment, the present invention relates to a method of treating angiogenesis-related diseases or lymphoangiogenesis-related diseases in a subject in need thereof, comprising the step of administering to said subject an agent selected from the group consisting of the polypeptide of the invention, the nucleic acid of the invention, the aptamer of the invention and the antibody of the invention.

### Screening Methods:

In a further aspect, the present invention relates to a method of screening a candidate compound for use as a drug for the treatment of angiogenesis-related diseases or lymphangiogenesis-related diseases in a subject in need thereof, wherein the method comprises the steps of: i) providing candidate compounds and ii) selecting candidate compounds that inhibits the between EMMPRIN and VEGFR2.

In a further aspect, the present invention relates to a method of screening a candidate compound for use as a drug for the treatment of angiogenesis-related diseases or lymphangiogenesis-related diseases in a subject in need thereof, wherein the method comprises the steps of:

(i) providing a EMMPRIN polypeptide, providing a VEGFR-2 (a polypeptide, providing a cell, tissue sample or organism expressing the EMMPRIN and VEGFR-2,

(ii) providing a candidate compound such as small organic molecule, antibodies, peptide or polypeptide,

(iii) measuring the activity of the binding between EMMPRIN and VEGFR-2,

(iv) and selecting positively candidate compounds that is able to inhibit the binding between EMMPRIN and VEGFR2.

Methods for measuring the binding between EMMPRIN and VEGFR-2 are well known in the art. For example, said methods involve measuring impaired association of EMMPRIN/VEGFR-2 on the EMMPRIN/VEGFR-2 cloned and transfected in a stable manner into a CHO cell line, human embryonic kidney (HEK) cell line or human endothelial cell line, measuring VEGF binding to its receptor VEGFR-2, measuring the proliferation of HMEC endothelial cells, or measuring ERK activation signaling in the presence or absence of the candidate compound. Tests and assays for screening and determining whether a candidate compound is an inhibitor of EMMPRIN/VEGFR-2 interaction are well known in the art. In vitro and in vivo assays may be used to assess the potency and selectivity of the candidate compounds to reduce EMMPRIN/VEGFR-2 activity. Activities of the candidate compounds, may be tested using isolated endothelial cells expressing EMMPRIN and VEGFR-2, CHO cell line, human embryonic kidney cell line (HEK) or human cell line cloned and transfected in a stable manner by the human EMMPRIN/VEGFR-2. Cells and endothelial cells expressing another VEGF receptor than VEGFR-2 may be used to assess selectivity of the candidate compounds.

The invention will be further illustrated by the following figures and examples.

However, these examples and figures should not be interpreted in any way as limiting the scope of the present invention.

### FIGURES:

FIG. 1. EMMPRIN/CD147 interacts with VEGFR-2 and VEGF in endothelial and tumor cells. A. VEGFR-2 and VEGF from HMEC and M10 cell lysates were immunoprecipitated (IP) with anti-VEGFR-2 and anti-VEGF antibody respectively; western blotting was performed using anti-EMMPRIN antibody. Non immune IgG was used as controls. Representative blots of three independent experiments are shown. B. In situ Proximity ligation assay (PLA) detection of EMMPRIN-VEGFR-2 and EMMPRIN-VEGF heterodimers (red dots). Negative controls without primary antibody are also shown. Nuclei were stained with DAPI (blue), magnification×63. Representative images of three independent experiments are shown. C. Direct interaction between the recombinant EMMPRIN and the recombinant VEGFR-2 in vitro. VEGFR-2 was first incubated with protein G beads prior to the addition of the recombinant EMMPRIN. Bound proteins were subsequently analyzed by Western blotting. Non-immune IgG served as a negative control and interaction between VEGF and VEGFR-2 served as a positive control. D. Cells (HMEC, MDA-MB-231 and M10) were transfected for 24 hours with EMMPRIN siRNA or scrambled control siRNA at 33nmol/L concentration, and then subjected to IP assays using antibodies against VEGFR-2 and VEGF. Western blotting was performed using anti-EMMPRIN antibody. Representative blots of three independent experiments are shown.

FIG. 2. EMMPRIN silencing inhibit EMMPRIN-VEGFR-2 interaction in endothelial and tumor cells. Cells were transfected with EMMPRIN siRNA or scrambled siRNA prior to in situ PLA for EMMPRIN-VEGFR-2 interaction. Cell nuclei were stained with DAPI (blue), magnification×63. The detected dimers (EMMPRIN/VEGFR-2) are represented as red dots. Representative images of three independent experiments are shown. Quantification of PLA signals was performed on ˜150 transfected cells per condition in three independent experiments; mean PLA signal/cell±SD are plotted. ***, P<0.0001.

FIG. 3. EMMPRIN interacts with pVEGFR-2 in vitro and in vivo. A. EMMPRIN interacts with pVEGFR-2 in HMEC endothelial cells and M10 tumor cells. In situ PLA for EMMPRIN/pVEGFR-2 was performed after VEGF stimulation (5 minutes, 50 ng/ml); red dots represent EMMPRIN-pVEGFR-2 interaction; nuclei are stained with DAPI (blue). Representative images of three independent experiments are shown. Quantification of PLA signals was performed on ˜150 transfected cells per condition in three independent experiments; mean PLA signal/cell±SD are plotted. **, P<0.001. B. EMMPRIN interacts with pVEGFR-2 in human cancer tissues. In situ PLA detection of EMMPRIN and pVEGFR-2 interaction in human melanoma tissues (M202 and M165) and in human breast cancer tissues (B132 and B18) using antibodies against EMMPRIN and pVEGFR-2. Nuclei were stained with DAPI (blue); phase contrast indicates cell contour (grey); the panels show high magnification (×40) to clearly visualize the PLA spots representing heterodimers. Representative photos of three independent experiments are shown.

FIG. 4. EMMPRIN knockdown in BLM xenografts inhibit EMMPRIN/pVEGFR-2 and VEGF/pVEGFR-2 interactions. Melanoma cell line BLM was transfected with EMMPRIN-miRNA (BLM-EMMPRIN-miRNA) or scrambled-miRNA (BLM-Scrambled-miRNA).EMMPRIN expression in 4 different clones was analyzed by: A. western blot (Western Blot was performed usinganti-EMMPRIN antibody normalized to actin; representative blots of three independent experiments); B. by qRT-PCR (means of relative expression to the reference gene PPIA of at least 3 independent experiments, error bars refer to 95% confidence intervals; *, P<(0.05)) and C. Invasion assay using a modified Boyden chamber was performed with clone 4 BLM-EMMPRIN-miRNA. Representative images of three independent experiments are shown. EMMPRIN/pVEGFR-2 interaction in experimental mouse model using EMMPRIN deficient tumor cells. Melanoma cell line BLM was transfected with EMMPRIN-miRNA (BLM-EMMPRIN-miRNA) or scrambled-miRNA (BLM-Scrambled-miRNA). D. Immunofluorescence analysis of pVEGFR-2 in xenograft tumors from Scrambled-miRNA or EMMPRIN-miRNA BLM cells 5 weeks after injection. Representative images of 10 primary tumors analysed are shown. E. EMMPRIN/pVEGFR-2 and VEGF/pVEGFR-2 interactions in EMMPRIN-silenced xenografts by in situ PLA. Nuclei were stained with DAPI, magnification x 40. Representative images of 10 primary tumors analysed are shown. Quantification of PLA signals was performed on ˜150 transfected cells per condition in three independent experiments; mean PLA signal/cell±SD are plotted. **, P≦0.001;***, P≦0.0001.

FIG. 5. EMMPRIN enhances VEGF-mediated VEGFR-2 activation (phosphorylation and homodimerization) in EMMPRIN silenced HMEC and M10 cells. Phosphorylation of VEGFR-2 by VEGF (5 minutes, 50 ng/ml) was assessed by: A. VEGFR-2 IP followed by immunoblotting for pVEGFR-2 and VEGFR-2 used as loading control (representative blots of three independent experiments are shown), and B. In situ PLA showing VEGF/pVEGFR-2 interaction.Quantification of PLA signals was performed on ˜150 transfected cells per condition in three independent experiments; mean PLA signal/cell±SD are plotted. ***, P≦0.0001 (magnification×63). C. In situ PLA detection of VEGFR-2 homodimers in HMEC endothelial cells. Nuclei were stained with DAPI, magnification×63. D. VEGF-induced downstream signalling by Phospho-Proteome profiling of EMMPRIN silenced HMEC cells. Total cell lysates (300 μg) were incubated with Human Phospho-Kinase Array membranes (containing 43 different kinases) (R&D systems) and developed by chemiluminescent system. Representative dots of selected kinases are shown.

FIG. 6. EMMPRIN is required in VEGF-induced VEGFR-2 cell migration. Cell migration was determined using a transwell system. EMMPRIN siRNA transfected cells (HMEC, MDA-MB-231 and M10) were seeded in 24-well/insert of Boyden chambers and treated with VEGF (50 ng/ml). After 24 hours of incubation, cells were fixed, stained with Diff-Quick and counted under a microscope. Columns indicate means of 3 independent experiments carried out in triplicate; and bars, SD*P<0.05.

FIG. 7. EMMPRIN/VEGFR-2 docking model. A. Best-energy docking model for the interaction between EMMPRIN monomer and VEGFR-2 D6-D7 model Interface residues are shown in ball & stick. B. Surface representation of EMMPRIN monomer residues, according to their electrostatic contribution to the VEGFR-2 D6-D7 binding energy. Interface residues are highlighted.

FIG. 8. EMMPRIN amino acid residues 195-199 are required for EMMPRIN/pVEGFR-2 interaction. EMMPRINNEGFR-2 interaction in BLM EMMPRIN-deficient cells transfected with EMMPRIN simple and double mutant constructs, control D136A and WT. After VEGFR-2 pull-downs, interaction with EMMPRIN was analyzed by Western blotting. Representative blots of three independent experiments are shown. In situ PLA using confocal microscopy shows red fluorescent spots which denote very close localization between EMMPRIN and pVEGFR-2. Fluorescence was markedly decreased with the double mutant Q195A/T199A. Nuclei were stained with DAPI, magnification×63. Representative images of three independent experiments are shown. Quantification of PLA signals was performed on ˜150 transfected cells per condition in three independent experiments; mean PLA signal/cell±SD are plotted.

FIG. 9. EMMPRIN amino acid residues 195-199 are required for VEGF-mediated VEGFR-2 activation. VEGF-mediated VEGFR-2 phosphorylation in BLM EMMPRIN-deficient cells transfected with EMMPRIN double mutant constructs, control D136A and WT. VEGFR-2 phosphorylation by VEGF (5 min) was analyzed by VEGFR-2 IP followed by immunoblotting for pVEGFR-2 and VEGFR-2. Representative blots of three independent experiments are shown. In situ PLA was performed to identify VEGF/pVEGFR-2 interaction with and without VEGF treatment. Nuclei are stained with DAPI, magnification×63. Representative images of three independent experiments are shown. Quantification of PLA signals was performed on ˜150 transfected cells per condition in three independent experiments; mean PLA signal/cell±SD are plotted. Comparing PLA signals between VEGF treated and non-treated showed significant difference for WT and control conditions; **, P≦0.001.

FIG. 10. Scheme of the modeling procedure followed in this work. The final models were obtained by a combination of EMMPRINNEGFR-2 D6-D7 docking, EMMPRIN/EMMPRIN docking and NMA-based conformational search. Those models compatible with the membrane attachement were selected.

FIG. 11. Models of EMMPRIN/VEGFR-2 interaction. A. a. Model of the interaction of EMMPRIN and VEGFR-2 on the membrane, based on our EMMPRIN/VEGFR-2 D6-D7 docking models, EMMPRIN dimer docking model, and inter-domain NMA-based conformational search. b. With a small rearrangement of VEGFR-2 D7 domains, this model is compatible with D7/D7 dimer x-ray structure. B. Proposed models for the role of EMMPRIN in VEGF-mediated VEGFR-2 activation. a. According to EMMPRIN/VEGFR-2 model, EMMPRIN could recruit VEGFR-2 dimers on the membrane surface, which can facilitate binding of VEGF to two VEGFR-2 monomers and hence favour D7/D7 orientation suitable for activation of VEGFR-2 intracellular domains (activation of intracellular signal is represented by a green flash). b. In the absence of EMMPRIN, VEGFR-2 monomers would be more spread on the membrane surface, so VEGF binding to two VEGFR-2 monomers (second step, marked by a dashed arrow) is less likely and therefore activation of intracellular signal would be smaller.

FIG. 12. VEGF-mediated VEGFR-2 phosphorylation in BLM EMMPRIN-deficient cells transfected with EMMPRIN mutant constructs D144A, Q1822, R184A, Q195A, T199A and WT. VEGF-mediated VEGFR-2 phosphorylation in BLM EMMPRIN-deficient cells transfected with EMMPRIN simple mutant constructs and WT. VEGFR-2 phosphorylation by VEGF (5 min) was analyzed by VEGFR-2 IP followed by immunoblotting for pVEGFR-2 and VEGFR-2. Representative blots of three independent experiments are shown. In situ PLA was performed to identify VEGF/pVEGFR-2 interaction (dots) with and without VEGF treatment. Nuclei are stained with DAPI, magnification×63. Representative images of three independent experiments are shown. Quantification of PLA signals was performed on ˜150 transfected cells per condition in three independent experiments; mean PLA signal/cell ±SD are plotted. Comparing PLA signals between VEGF treated and non-treated showed significant difference for WT and control conditions; **, P≦0.001.

FIG. 13. Effects of EMMPRIN peptide inhibitor (PI) on the proliferation of HMEC endothelial cells (EC).

FIG. 14: Effects of EMMPRIN PI on the invasiveness of M10 melanoma cells.

FIG. 15: Effects of PI on EMMPRIN/VEGFR-2 interaction disruption.

FIG. 16: Effects of PI on ERK activation signalling.

FIG. 17: show the sequence of EMMPRIN, the domain responsible for the interaction (in bold) with VEGFR-2 and the location of peptide P1 in said sequence (bold and underlined).

### EXAMPLES

### Example 1

Material & Methods

Cell Culture

Human microvascular endothelial (HMEC) cells line derived from dermal microvasculature (T. Lawley, Emory University, Atlanta, GA) were maintained in MCDB-131 medium (Gibco, Invitogen) with 10% fetal bovine serum (FBS) (Invitrogen), 2 ml glutamine (Invitrogen), 10 ng/ml endothelial growth factor (Upstate Biotechnology/Millipore), and 1 μg/ml hydrocortisone (Sigma-Aldrich). Primary melanoma M10 cells, established from patient primary nodular melanoma were maintained in RPMI medium (Gibco, Invitogen) with 10% FBS, Hepes 1 M, pyruvate Nas, and glutamine (Invitrogen). Human breast carcinoma MDA-MB-231 cells were maintained in Dulbecco's modified Eagle's medium (DMEM) (Gibco, Invitogen) with 10% FBS (Invitrogen) and 2m1 glutamine (Invitrogen). Melanoma BLM cells (American Type Culture Collection (ATCC Manassas, Va.)) were maintained in DMEM containing 4.5 g/l glucose, 10% FBS, 100 U/ml penicillin and 100 mg/ml streptomycin. HEK293T cells (ATCC) were cultured in DMEM medium (Gibco, Invitogen) supplemented with 10% FBS (Invitrogen), 100 U/ml penicillin, 100 mg/ml streptomycin and 2 ml glutamine (Invitrogen).

Immunoprecipitation and Western blotting Analyses

Cells treated or not with human recombinant VEGF (50 ng/ml; R&D Systems) for 5 minutes at 37° C. were harvested, washed with PBS and lysed in extraction buffer (TBS-Nonidet P-40 solution comprising 50 mM Tris buffer pH 7.5, 150 mM NaCl, 0.5% Nonidet P-40, 5 mM NaF and 0.2 mM Na3VO4 in the presence of Complete Protease Inhibitor Cocktail (Roche)). For immunoprecipitation, cell lysates were incubated with antibodies against targeted proteins and Protein G-sepharose beads (Sigma). Immunoprecipitated proteins were subjected to sodium dodecyl sulphate-polyacrylamide gel electrophoresis then transferred to Nitrocellulose membranes and probed with anti-EMMPRIN mAb (555961, BD-Pharmingen), anti-VEGF (C-1) mAb (Sc-7269, Santa Cruz), anti-VEGFR-2 rabbit pAb (Sc-504, Santa Cruz-) or anti-pVEGFR-2 (Tyr 1175) rabbit mAb (2478, Cell Signaling). The proteins were visualized with ECL reagent (Pierce), and their expression was normalized relative to total cell lysate protein concentration.

In situ Proximity Ligation Assay (PLA)

In situ PLA was used to assess protein-protein close proximity. Cells grown on 8-well culture slides (Lab-tek chamber slides (Nunc, #154534)), were immediately fixed and subjected to in situ PLA using the Duolink Detection kit (Olink Bioscience, Sweden) according to the manufacturer's instructions. Briefly, after blocking slides were incubated with mouse anti-EMMPRIN (1:250, 555961, BD, Pharmingen), rabbit anti-VEGFR-2 (1:50; Santa cruz), mouse anti-VEGF (1:200; Santa cruz) or rabbit anti-pVEGFR-2 (Tyr 1175) (1:100; Cell Signalling) primary antibodies. PLA minus and PLA plus probes (containing the secondary antibodies conjugated with oligonucleotides) were added. For VEGFR-2 homodimers detection, primary antibody was prepared using the Probemaker kit (OLINK, Bioscience) according to manufacturer's instructions: 1 mg/ml of monoclonal antibody (affinity purified through a protein G column) was independently conjugated to each of a pair of oligonucleotides to generate plus and minus PLA probes. Thereafter, further oligonucleotides are added, allowed to hybridize to the PLA probes, and ligase joins the two hybridized oligonucleotides to a closed circle. The DNA is then amplified (rolling circle amplification), and detection of the amplicons was carried by a fluorescently labeled probe (Detection Kit 563). Protein complexes were visualized in a laser-scanning confocal microscope (Leica-Lasertechnik) as bright fluorescent signals. For PLA analysis of frozen tumor tissues, cryosections were fixed with 4% Paraformaldehyde for 15 min, and in situ PLA assay was performed as described above for cultured cells. Fluorescent and phase contrast images were taken. Negative controls without primary antibody were performed.

Small Interfering RNA Transfection

siRNA for EMMPRIN (IDs: 147251 and 215973) or scrambled siRNA oligos (Ambion/Applied-Biosystems, France) were transfected into cells by using the Lipofectamine-2000 (Invitrogen). Cells were then incubated for 24h prior to treatment with VEGF and were then analyzed by Co-immunoprecipitation, Western Blotting, in situ PLA, cell migration and phospho-kinase array.

EMMPRIN Stable Knockdown

In order to knockdown EMMPRIN expression in BLM cell line, lentivirus-based miRNA was used. MicroRNA sequence EMMPRIN-miRNA targeting human EMMPRIN was selected with Invitrogen Block-iTRNAi Designer software (www.invitrogen.com/rnai), and srambled-miRNA (Invitrogen) was used for the negative control [20].The U6 promoter-miRNA-Ubiquitin promoter-mCherry cassette was cloned into the BamHI and Xhol sites in the lentiviral vector pTK431[22]. The vector plasmids (either pTK431-EMMPRIN-miRNA or pTK431-scrambled-miRNA), together with the packaging construct plasmid pDNRF and the envelope plasmid pMDG-VSVG, were cotransfected into HEK293T cells to produce the viral particles [22, 23]. The viral titres were determined by p24 antigen measurements (KPL, Lausanne, Switzerland). BLM cells were plated in a 24-well plate at a density of 10.000 cells/well in culture medium. At 60% of confluent, LV-EMMPRIN-miRNA (121 ng/μL of P24) or LV-scrambled-miRNA (97 ng/μL of P24) was added in 100 μl of complete culture medium without FBS. After overnight incubation with the vectors, medium was refreshed and cells were allowed to growth. For determination of transduction efficiencies, transduced cultures were analyzed by cell sorting with a FACS ARIAIII (Becton-Dickinson, San Jose, Calif., USA), real-time PCR, Western blotting and invasion assay.

Real-Time Quantitative PCR (qRT-PCR)

Total RNA was extracted from BLM-scrambled-miRNA or BLM-EMMPRIN-miRNAcells using Trizol reagent (Invitrogen). RNA quantity and quality were assessed using the Nanodrop-ND-1000 (Nanodrop Technologies, Wilmington). First-strand cDNA was synthesized using a High-Capacity cDNA Archive Kit (Applied-Biosystems) according to the manufacturer's protocol. EMMPRIN primers were specifically designed (Eurogentec, Belgium). Transcript levels were measured by qRT-PCR using Perfect Master Mix-Probe (AnyGenes, France) on LightCycler-480 (Roche) according to the manufacturer's protocol. The transcript levels were normalized to the housekeeping PPIA (peptidylprolylisomerase A) transcripts.

Immunofluorescence, Confocal Microscopy

Sections of BLM-Scrambled-miRNA and BLM-EMMPRIN-miRNA derived tumor tissues were fixed and incubated with primary anti-pVEGFR-2 antibody (Cell signaling) followed by Alexa Fluor 488 fluorescently conjugated secondary antibody (Molecular Probes). DAPI was used for nuclear counterstaining. Confocal images were taken with a Leica inverted confocal microscope (Leica Lasertechnik, Heidelberg).

Animal Experiment

Mouse experiments were conducted according to French veterinary guidelines and those formulated by the council of Europe for experimental animal use (L358-86/609EEC). Female 5-week-old nude/c mice (Janvier) were injected subcutaneously with 5×106 stably transfected BLM-EMMPRIN-miRNA or BLM-scrambled-miRNA cells (n=10 mice for each cell line). Five weeks later, all mice were sacrificed by cervical dislocation and tumors were resected and stored in liquid nitrogen prior to in situ PLA and immunofluorescence assays.

Migration and Invasion Assays

The in vitro migration (on uncoated filters) and invasion (on coated filters with matrigel, BD Bioscience) were performed using a modified Boyden chamber [24] in 24-well plates and 8-mm pore filter inserts (BD Bioscience). After 24h of incubation, cells were fixed, stained with crystal violet 0.5% and counted under a light microscope.

Human Phospho-Kinase Array

The human phospho-Kinase Array Kit (Proteome Profiler Array, ARY003, R&D Systems) was used to detect relative levels of phosphorylation of 46 kinase phosphorylation sites, according to the manufacturer's instructions, using total cell lysates of EMMPRIN or scrambled siRNA transfected HMEC cells treated or not with 50 ng/ml VEGF. Briefly, cell lysates diluted to 300 μg/mL of protein in a detergent- urea and phosphatase inhibitor-containing solubilizing buffer (R&D Systems) were incubated with the arrays overnight at 4° C. After washing unbound material, membranes were incubated with a cocktail of phosphosite-specific, biotinylated antibodies, and phosphorylated kinases were detected with streptavidin-horseradish peroxidase. Signals were revealed with a chemiluminescent substrate kit (ECL Dura Thermo Scientific, 34076). Independent experiments were performed in duplicates.

Modelling: General Consideration

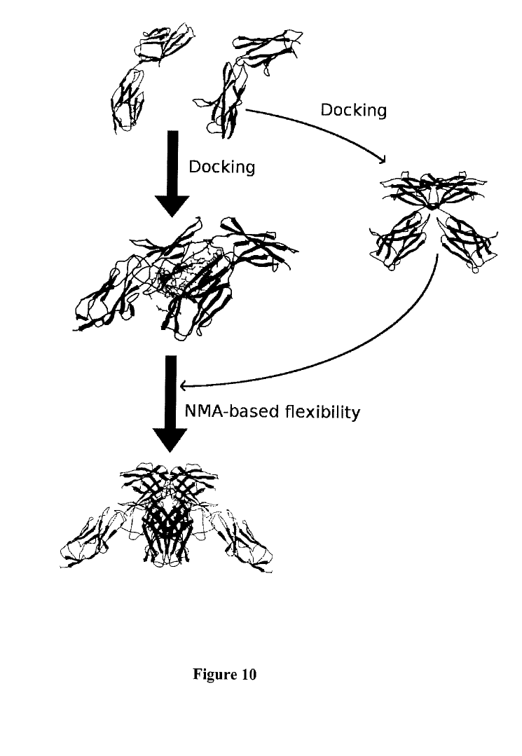

We modelled EMMPRIn/VEGFR-2 association by using homology-based modeling, computational docking, and conformational sampling by normal mode analysis (NMA) (FIG. 10). On the one side, we built a model for VEGFR-2 D6-D7, and we docked it to EMMPRIN x-ray structure. The EMMPRIN/VEGFR-2 docking model was later confirmed by site-directed mutagenesis. We also used docking to build models for EMMPRIN dimerization. The docking models obtained for EMMPRIN/VEGFR-2 and EMMPRIN dimer, in combination with NMA-based sampling, were compatible with membrane attachment, and with D7 x-ray dimers. We tried other docking combinations but could not find any better model.

Homology-Based Modeling of VEGFR-2 D6-D7

We used Modeller 8v1 [25] to model the structure of VEGFR-2 D6-D7 domains from 1F97 PDB template structure, with 24% of sequence identity and selected by FUGUE server (http://tardis.nibio.go.jp/fugue/prfsearch.html) [26] as the best homologous topology. The D7 coordinates in the model were replaced by the known x-ray structure (3KVQ PDB). The resulting D6-D7 construct (in particular the linker between D6 and D7 domains) was finally refined by Modeller 8v1.

Computational Docking

Computational docking was performed by combining the 10,000 output solutions from FTDock 2.0 [27] and the 2,000 ones from ZDock 2.1 [28]. The resulting 12,000 solutions were then scored by pyDock [29].

Energetic Analysis of Interaction Model

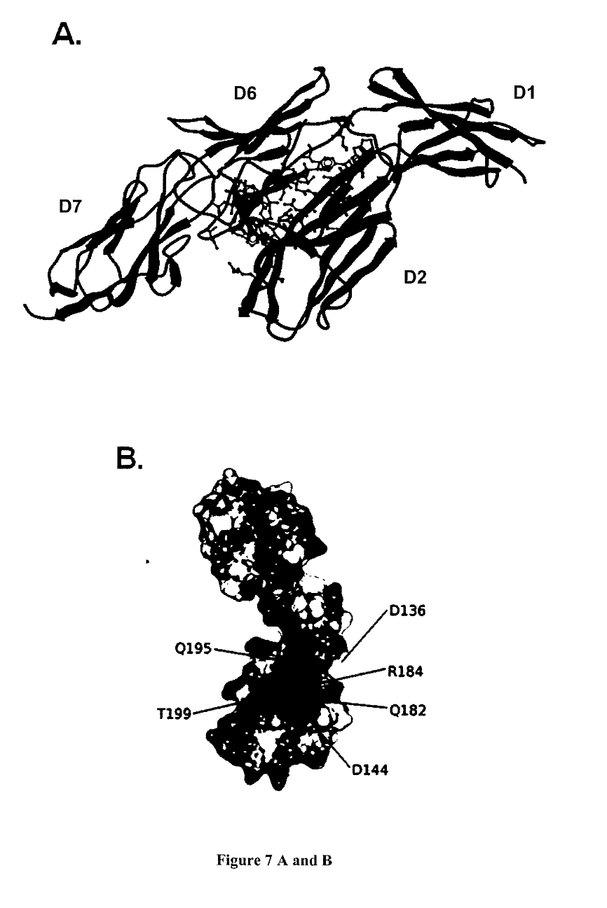

The best docking model for EMMPRIN D1-D2NEGFR-2 D6-D7 was energetically minimized using Tinker (http://dasher.wustl.edu/tinker/). The global binding energy had a clear electrostatic contribution. The binding energy per residue was calculated for this minimized structure using pyDock [29]. Residues with the highest electrostatic contribution were further considered for selecting mutant candidates (FIG. 7B).

NMA-Based Conformational Sampling

We used iMC module from iMOD [30] (with a maximum amplitude of 6 Å) for conformational sampling of extracellular EMMPRIN and VEGFR-2 D6-D7 model. With this method, we generated 100 conformations independently for EMMPRIN and VEGFR-2 D6-D7 monomers. These NMA-based EMMPRIN conformations were combined to produced new EMMPRIN dimer models, built based on the main dimer interface described by the EMMPRIN/EMMPRIN docking model. These EMMPRIN dimer models were combined with the NMA-based VEGFR-2 D6-D7 conformations, based on the main interface described by the EMMPRIN/VEGFR-2 D6-D7 docking model. This generated 10,000 (2:2) EMMPRIN/VEGFR-2 D6-D7 models. Finally, when considering cell membrane, we obtained a total of 32 models in which C-term regions from all molecules were located approximately in the same plane (to represent the attachment to the membrane) and with more than 80% of the atoms located in one side of the defined plane (in order to disregard strong steric clashes with the cell membrane).

Site Directed Mutagenesis

EMMPRIN residues (Asp144, Gln182, Arg184, Gln195, Asp136 and Thr199) involved in the interaction between EMMPRIN and VEGFR-2 were mutated to Alanine using>>Geneart Site-Direct Mutagenesis system—(Lifetechnologies) according to the manufacturer's instructions. The following mutations were made in the PCRII vector containing EMMPRIN full length cDNA (PCRII-EMMPRIN) [10]. Briefly, the mutagenesis reactions were performed using Platinum Taq DNA polymerase (Lifetechnologies), with specifically designed mutagenesis primers and cycling conditions as follows: 37° C. for 20 minutes, 94° C. for 2 minutes followed by 18 cycles of 94° C. for 20 seconds, 57° C. for 30 seconds and 68° C. for 2.5 minutes; and finally 1 cycle of 68° C. for 5 minutes. Each mutagenesis product was transfected into chemically competent DH5 □T1R E.coli (Lifetechnologies) and grown at 37° C. overnight. Colonies were selected and screened for the correspondant mutation at each site by DNA sequencing. PCRII-EMMPRIN wide type (WT) and mutated were transfected into BLM-EMMPRIN-miRNA cells using the Lipofectamine-2000 (Invitrogen).

Statistical Analysis

Data are presented as the mean values±SD. Mann-Whitney test was used to evaluate differences between groups. Data were considered statistically significantly different for P value <0.05. All statistical tests were two-sided. Analyses were performed using Prism 6 (GraphPad Software Inc, La Jolla, Calif.).

Results

EMMPRIN/CD147 Interacts with VEGFR-2 in its Non-Phosphorylated and Phosphorylated Forms in Endothelial and Tumor Cells In Vitro and In Vivo

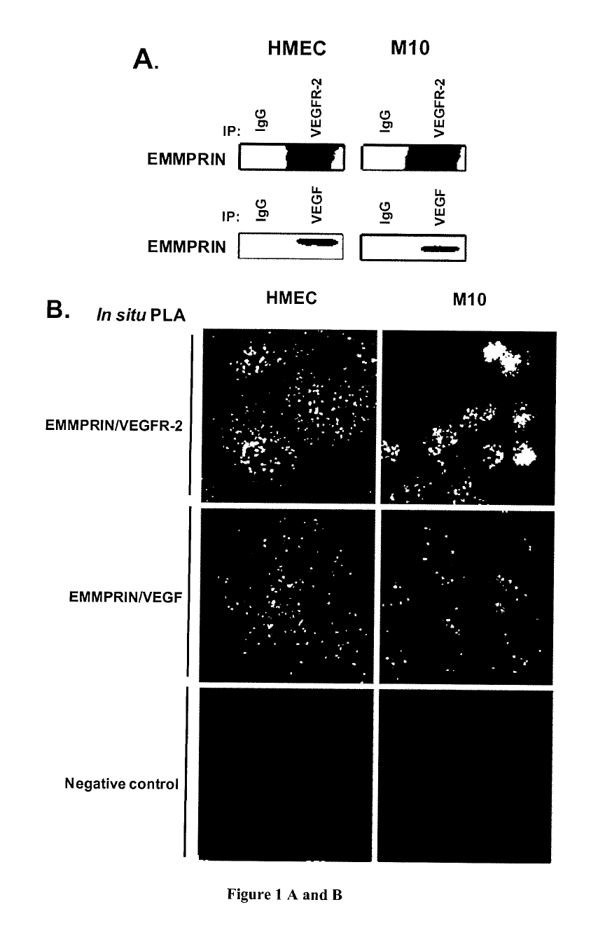

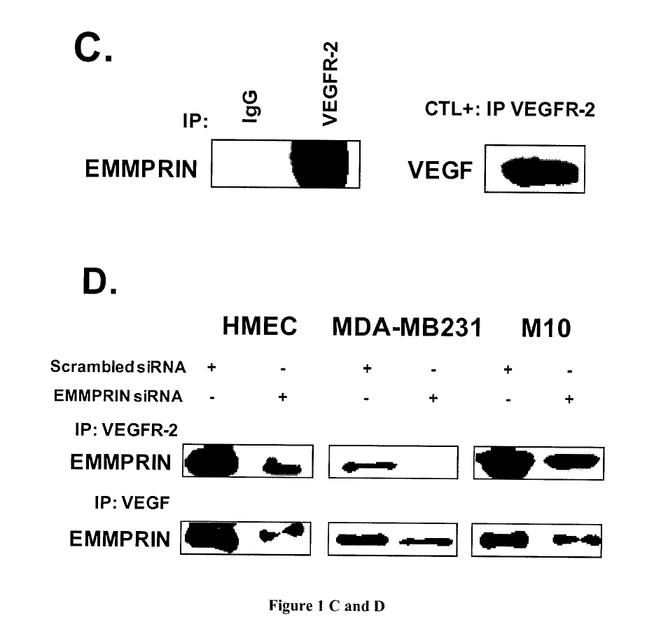

The potential interaction between EMMPRIN and VEGFR-2 was investigated by immunoprecipitation (IP) assays in endothelial cells HMEC and melanoma cells M10. Complex formation was identified by the immunoprecipitation of either VEGFR-2 or VEGF followed by EMMPRIN immunoblotting (FIG. 1A). IgG was used as a negative control. The fluorescent red spots observed using in situ proximity ligation assay (PLA) (FIG. 1B) and confocal microscopy, a method which highlights protein/protein closely colocalized in cells, confirmed the proximity between EMMPRIN and VEGFR-2, and to a lesser extent between EMMPRIN and VEGF, at the cell surface.

To further investigate whether EMMPRIN interacts directly with VEGFR-2 in a cell-free system, we performed pull-down assays using recombinant EMMPRIN and recombinant VEGFR-2. Our results show that VEGFR-2 bound specifically to EMMPRIN and to the same extend as to VEGF, used as a positive control (FIG. 1C).

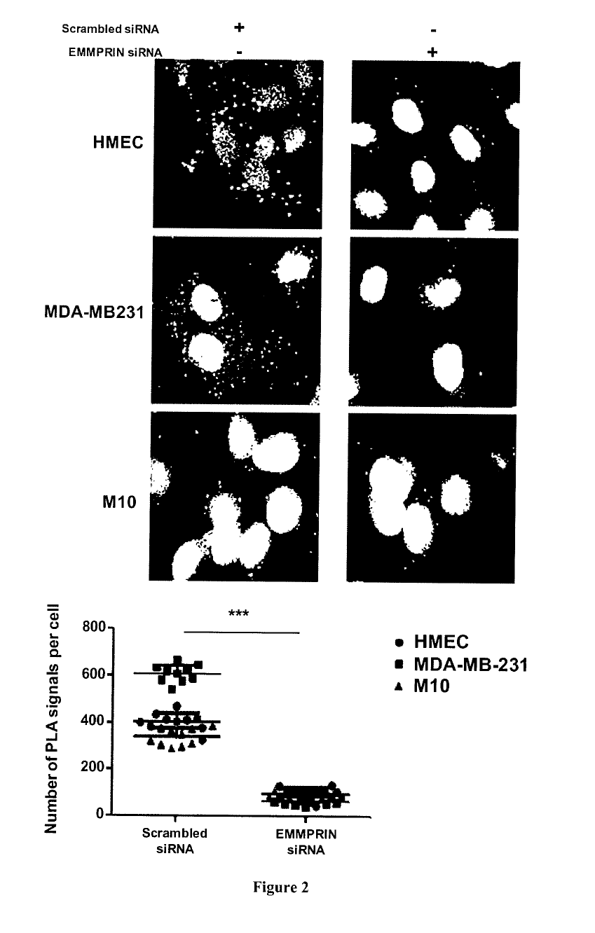

The specificity of EMMPRIN/VEGFR-2 interaction was demonstrated by the decrease in the immunoprecipitated (IP) complex when EMMPRIN expression was silenced using siRNA strategy (FIG. 1D). This was confirmed by PLA assay showing a large decrease in the number of red dots of cells transfected with EMMPRIN siRNA in both endothelial and tumor cells compared with its corresponding scrambled siRNA (FIG. 2). Similar results were obtained with BLM melanoma cells (not shown).

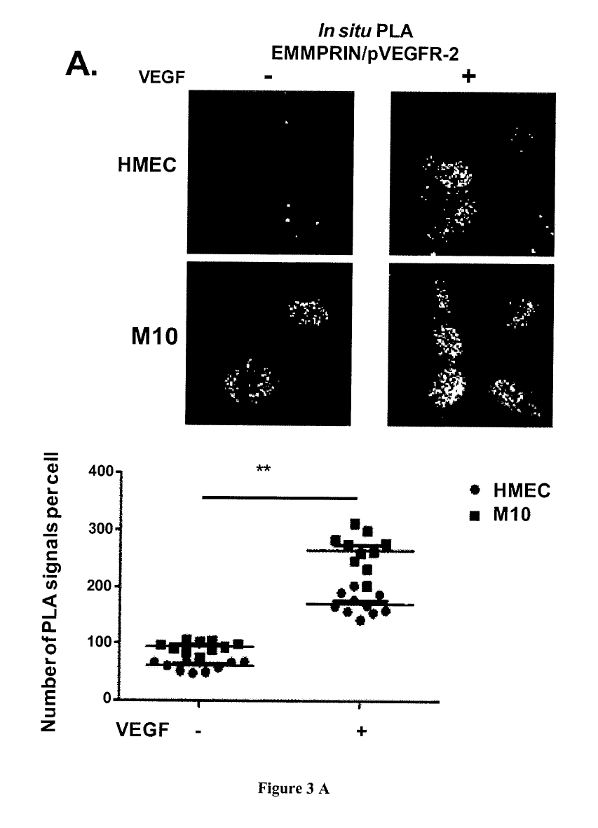

We have next shown that EMMPRIN also interacted with the active form of VEGFR-2 and this interaction was enhanced after VEGF treatment of endothelial as well as melanoma cells. EMMPRIN/pVEGFR-2 heterodimers are visualized by PLA red dots in FIG. 3A. Importantly, intense clustering pattern of these EMMPRIN/pVEGFR-2 heterocomplexes were also observed in human breast cancer (n=11) and melanoma (n=15) tissues (FIG. 3B) demonstrating the implication of EMMPRIN/pVEGFR-2 interactions in vivo.

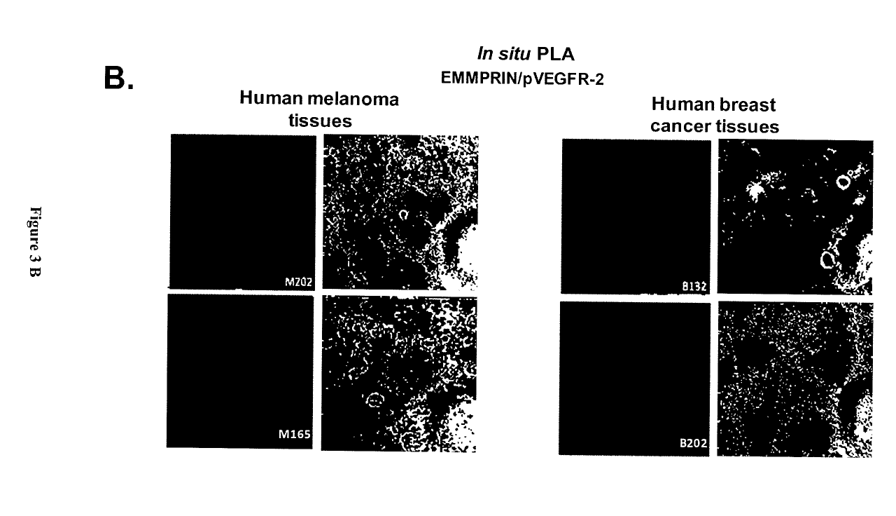

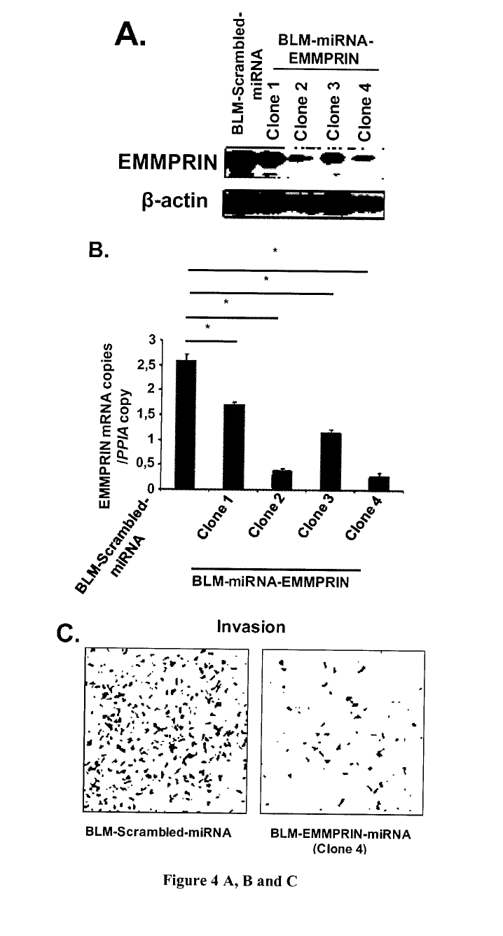

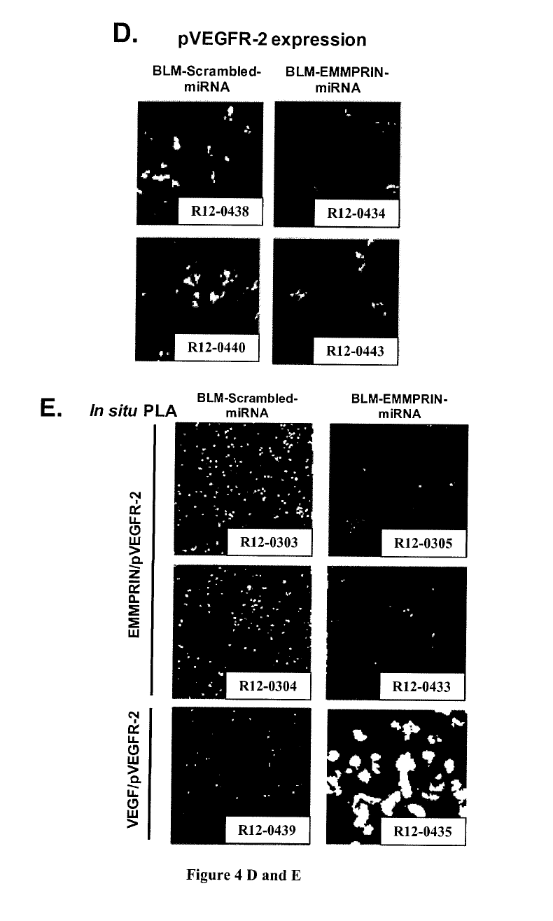

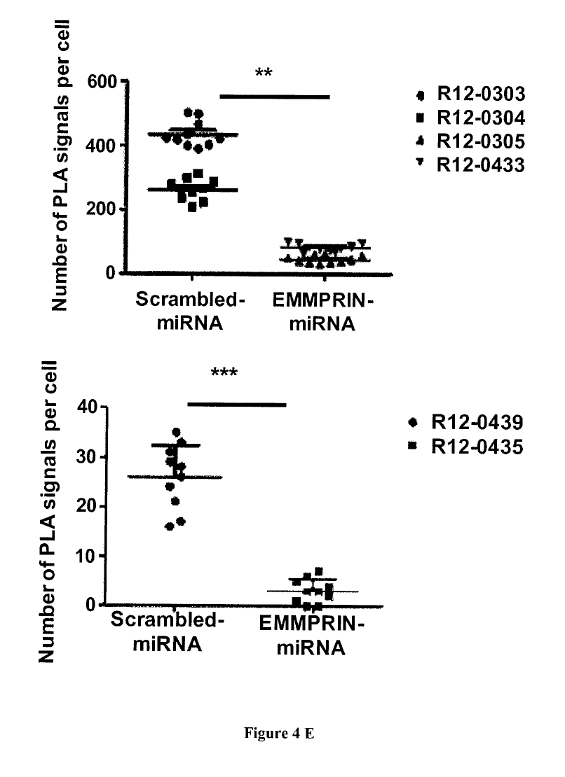

To investigate the role of EMMPRIN in VEGF/pVEGFR-2 interaction in vivo, we generated melanoma BLM cells with stable knockdown of EMMPRIN (EMMPRIN-miRNA) for injection in nude mice. The 4 clones of BLM-EMMPRIN-miRNA analyzed showed a decrease in EMMPRIN expression (protein and mRNA) in comparison to BLM-srambled-miRNA. This decrease was greatest in clone 2 and 4 which also correlated with the lowest invasive capacity of these clones; clone 4 was chosen for the in vivo studies (FIG. 4 A, B and C).

Analysis of tumor xenograft sections showed a decrease in pVEGFR-2 immunostaining and in EMMPRIN/pVEGFR-2 interaction in EMMPRIN knockdown tumors (BLM-EMMPRIN-miRNA), compared to control tumors (BLM-scrambled-miRNA). Importantly, this was associated with a significant decrease in VEGF/pVEGFR-2 interaction (FIG. 4 D and E).

EMMPRIN is Required for VEGF-Mediated VEGFR-2 Activation and Downstream Signalling

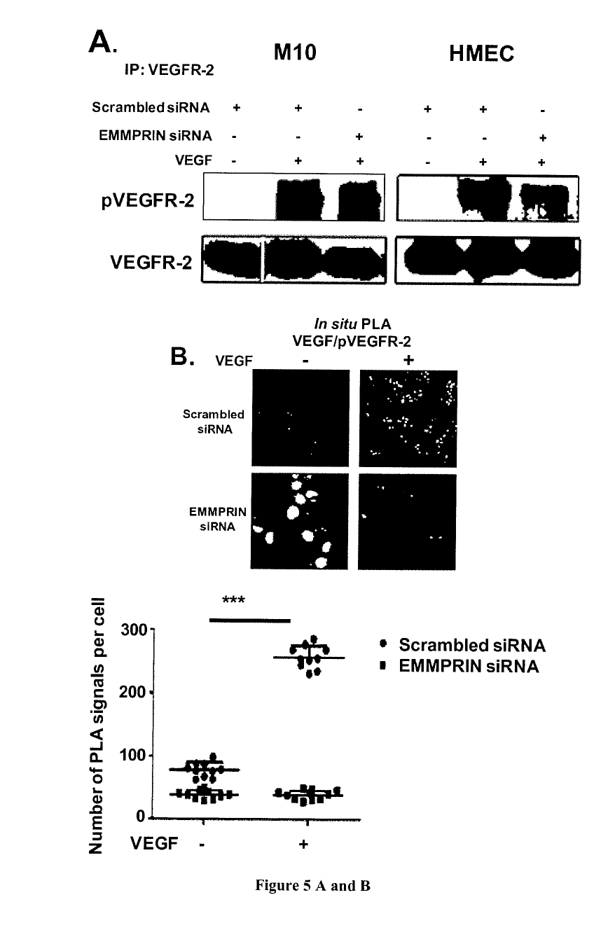

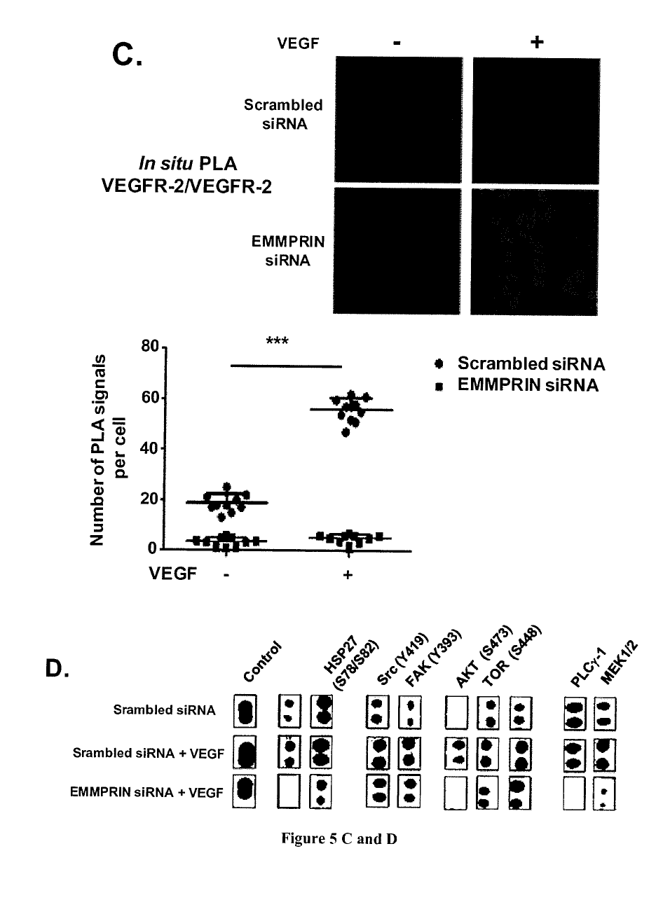

We next investigated the potential role of EMMPRIN in the activation of VEGFR-2 by its VEGF ligand. Immunoprecipitation of VEGFR-2 followed by immunoblotting with pVEGFR-2 antibody have shown that EMMPRIN knockdown by siRNA decreased VEGFR-2 phosphorylation mediated by VEGF, in both endothelial and tumor cells (FIG. 5A). Furthermore, PLA experiments have shown that the reduced activation of VEGFR-2 observed with EMMPRIN inhibition was associated with a decrease in both VEGF/pVEGFR-2 interaction (FIG. 5B) and VEGFR-2 homodimerization (FIG. 5C) (the decrease was even greater in the presence of VEGF), demonstrating the importance of EMMPRIN in VEGFR-2 phosphorylation mediated by VEGF (FIG. 5B). Phospho-Proteome Profiler Array analysis showed that certain VEGF-induced downstream signals were inhibited upon EMMPRIN silencing (FIG. 5D). Of interest, the activation of p38 and its downstream HSP27 involved in migration were impaired, but not that of the kinases FAK or Src. In addition, the PLCy-1/MEK1/2 pathway involved in cell proliferation was also affected. These findings suggest a role of EMMPRIN in VEGF signalling.

The functional consequence of this regulation was demonstrated by a decrease in cell migration observed in EMMPRIN siRNA transfected cells that was not restored after VEGF treatment.

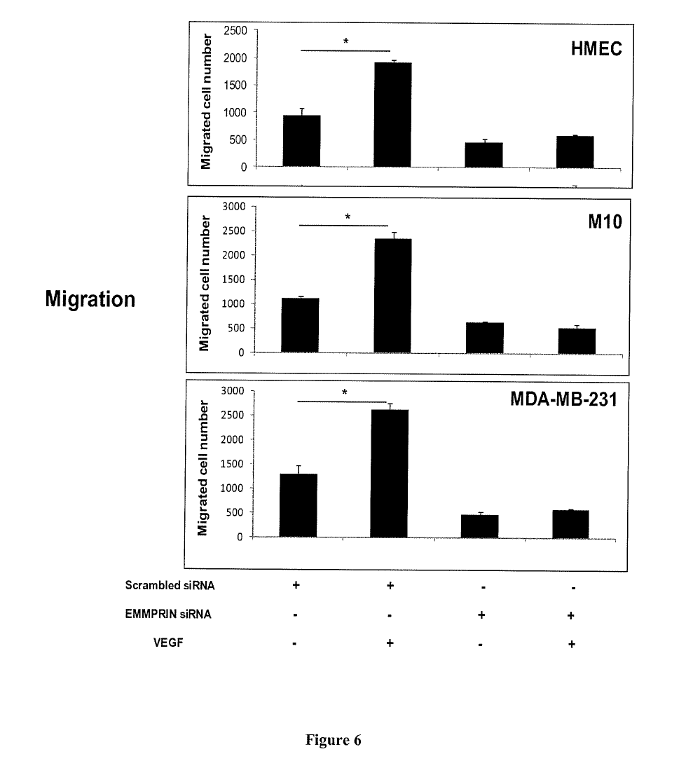

Cell migration was investigated using modified Boyden chamber assays. Migration of HMEC, M10 and MDA-MB-231 cells in which EMMPRIN was inhibited with siRNA was measured in the presence or not of VEGF. EMMPRIN silencing greatly inhibited cell migration stimulated by VEGF in the studied models (FIG. 6).

A Molecular Model for EMMPRIN/VEGFR-2 Direct Interaction

The above findings indicate that EMMPRIN interacts with VEGFR-2 to enhance its activation by VEGF, therefore we aimed to build a structural model for EMMPRIN/VEGFR-2 complex that could explain the experimental results. Modeling this system presented several major challenges such as: i) it was not known whether EMMPRIN interacts with VEGFR-2 as monomer, dimer or oligomer; ii) the interacting molecules are expected to have significant interdomain flexibility; and iii) it was not known which VEGFR-2 domain could be involved in the interaction. Therefore we performed a comprehensive modeling strategy using all the available structural information for the possible interacting domains (see Methods). The best docking model was obtained by using one of the EMMPRIN monomers from the x-ray structure and a two-domain construct of VEGFR-2 D6-D7 domains. Interestingly, the lowest-energy binding mode obtained by docking simulations was compatible with membrane binding, and had the majority of interactions between EMMPRIN D2 and VEGFR-2 D6 domains (FIG. 7).

Site-directed mutagenesis confirm EMMPRIN/VEGFR-2 interaction model The contribution of hot spots residues to EMMPRIN/VEGFR-2 interaction according to the above described model was examined by computational analysis. The EMMPRIN/VEGFR-2 interface according to this model is highly electrostatic (FIG. 7B). Based on the model, and considering the residues with highest electrostatic binding energy that were not involved in important intra-domain interactions, the following EMMPRIN mutants were constructed in order to validate the binding interface site: D144A, Q182A, R184A, Q195A, T199A (FIG. 7). We also generated Q182A/R184A and Q195A/T199A double mutants (see Methods). D136A was defined as a negative control, since according to the model this residue should not be involved in the interaction.

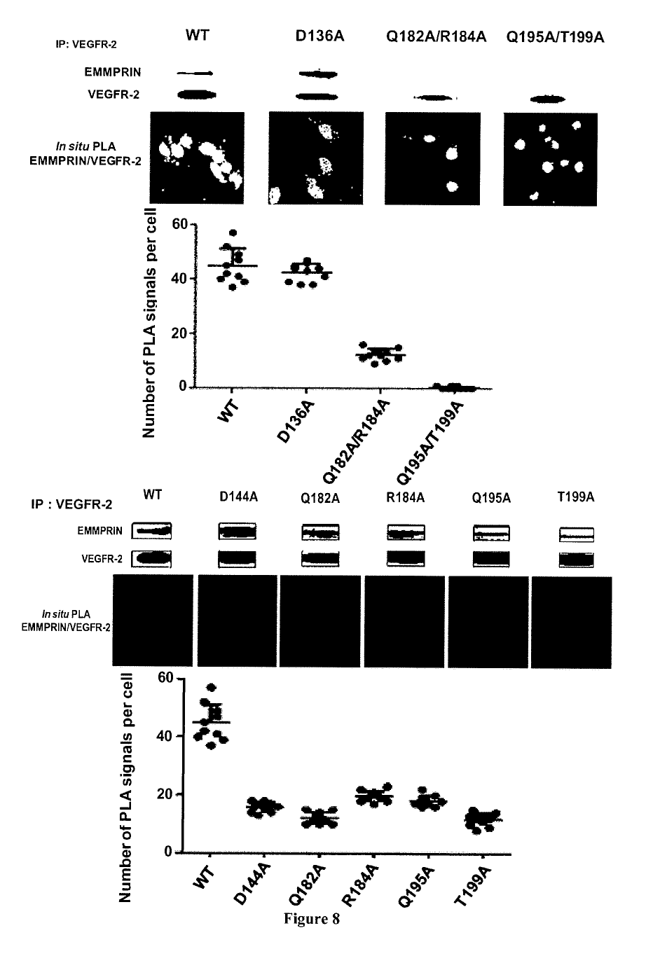

We first transfected BLM-EMMPRIN-miRNA cells with EMMPRIN full length cDNA (wide type: WT) or mutated on the following residues: D136A, D144A, Q182A, R184A, Q195A, T199A, Q182A/R184A and Q195A/T199A. EMMPRIN/VEGFR-2 binding was evaluated by immunoprecipitation using an antibody directed against VEGFR-2 (FIG. 8). Results show that both the single and the double mutants reduced EMMPRIN/VEGFR-2 binding to a varying degrees but the greatest reduction was observed with the double mutant Q195A/T199A pointing to the importance of this site in the interaction. By contrast, the negative control D136A mutant had no detectable effect on EMMPRIN/VEGFR-2 binding.

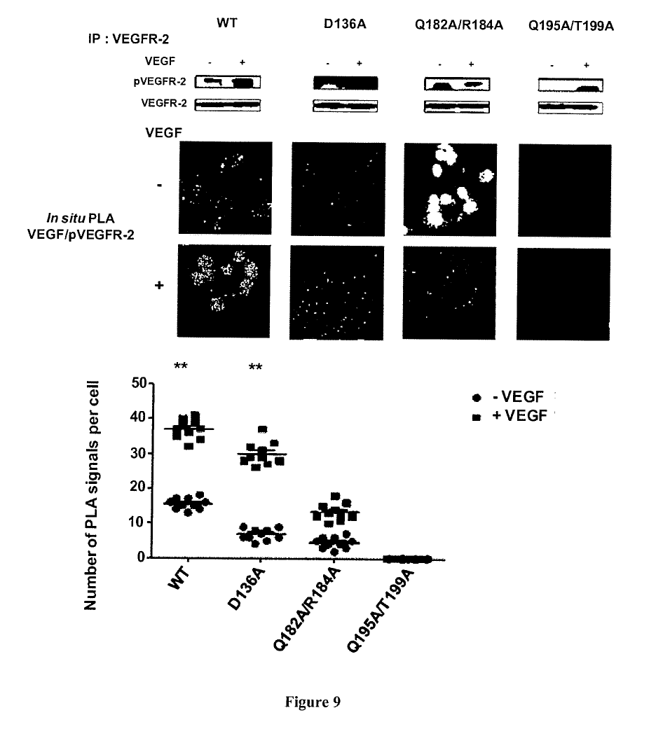

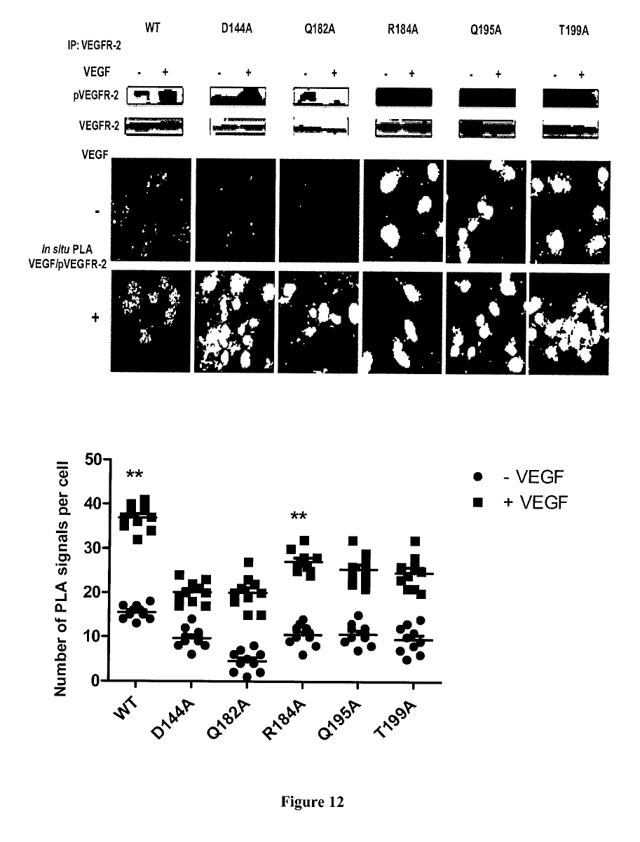

The role of these EMMPRIN residues on the activation of VEGFR-2 by its ligand VEGF was investigated by studying the binding behaviour of the EMMPRIN mutants towards VEGF-induced VEGFR-2 activation. There is a total inhibition of pVEGFR-2 activation by VEGF with the double mutant Q195A/T199A while the other single mutants had lower effects (FIGS. 9 and 12). This is consistent with the VEGF/pVEGFR-2 interaction results obtained by in situ PLA.

Altogether, our results uncovered a novel mechanism by which EMMPRIN regulates VEGFR-2 activation by direct binding, modulating its downstream signalling and functional consequences.

Discussion

EMMPRIN/CD147 has been reported to play crucial roles not only in matrix proteolysis and tumor invasion but also in angiogenesis [8]. We hypothesized that a possible link between EMMPRIN and VEGFR-2 may exist since both these membrane receptors localized on endothelial and tumor cell surface are involved in common functional properties, notably angiogenesis. In this study, we uncovered a novel function of EMMPRIN as a coreceptor of VEGFR-2, as it directly interacts with it and regulates its activation, signalling and functional consequences. Furthermore, in both endothelial and tumor cells, EMMPRIN enhanced VEGF-induced VEGFR-2 phosphorylation, downstream signalling of the VEGF-induced pathway, and consequently cell migration. Our results show that EMMPRIN/VEGFR-2 interaction involves a binding site located in the extracellular domain of EMMPRIN which contains the amino acids 195/199 located very close to the cell membrane, since mutating this site blocked the interaction. In addition, our in vivo studies showed that VEGF/pVEGFR-2 interaction is significantly impaired in mice injected with EMMPRIN-miRNA transfected BLM.

It is interesting to note that high expression of EMMPRIN in human renal cancer was reported to be involved in sunitinib (VEGFR inhibitor) resistance [20]. As EMMPRIN is highly expressed in cancer its interaction with VEGFR-2 may represent one underlying mechanism of this resistance.

In order to determine whether EMMPRIN/VEGFR-2 binding could explain the enhancement in VEGF-mediated VEGFR-2 dimer formation and VEGFR-2 activation by EMMPRIN, we explored the possible oligomerization state of EMMPRIN when interacting with VEGFR-2 in our model. It has been reported that EMMPRIN can dimerize in cis (both monomers in the membrane of the same cell), through the domain D1, but the structure of the dimer is not known. Therefore, we modeled the dimer of EMMPRIN extracellular domains by docking two monomers from the x-ray structure (see Methods). Interestingly, the lowest-energy docking solution is symmetric and would be compatible with membrane attachment (FIG. 10). It should be noted that it was impossible to find a dimer conformation that fully satisfied the recently reported mutational data on EMMPRIN dimerization in solution, which suggests that membrane attachment imposes additional structural restraints to EMMPRIN dimerization [21].

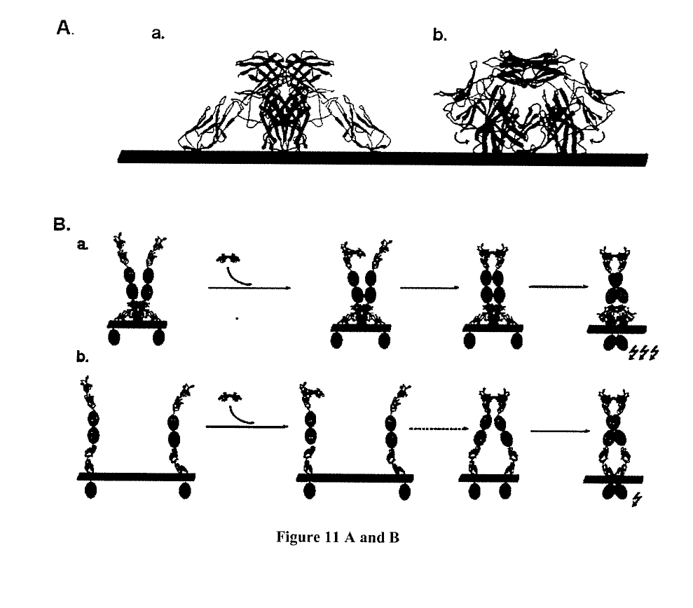

We combined the above described models obtained for EMMPRIN/VEGFR-2 complex and EMMPRIN dimer, allowing interdomain flexibility with NMA (see Methods), and found many possible rearrangements that are compatible with membrane attachment (see an example in FIG. 11A). Interestingly, with a small interdomain rearrangement, the D7 domains could form a dimer as in VEGFR-2 D7 x-ray structure, keeping compatibility with membrane attachment (FIG. 11B).

The model showing in FIG. 7A for EMMPRIN/VEGFR-2 interaction suggests that EMMPRIN can stabilize a VEGFR-2 dimer in which D7 domains are not in the expected proximity to activate VEGFR-2 intracellular domain. This is compatible with our findings that EMMPRIN can dimerize VEGFR-2 (FIG. 5C) but cannot activate it by itself (FIG. 5A). However, we also found that EMMPRIN enhances VEGF-mediated VEGFR-2 dimerization and thus activation of intracellular signalling (FIG. 5 A and C). A possible model for this is shown in FIG. 11B. When EMMPRIN is present, it can help to recruit VEGFR-2 molecules and form dimers, so when VEGF is added, its probability of binding two VEGFR-2 monomers increases. The binding of VEGF to two VEGFR-2 monomers will facilitate the D7 domains to form a dimer so that the intracellular domains can adopt a suitable orientation that triggers auto-phosphorylation and thus activation of the intracellular signalling (FIG. 11B a). However, in the absence of EMMPRIN, unligated VEGFR-2 molecules are not necessarily located in the proximity of each other and therefore VEGFR-2 dimer formation after VEGF binding, although possible, would be a limiting step that could make the intracellular signalling activation less efficient (FIG. 11B b).

Taken together, these results provide evidence that EMMPRIN is a novel coreceptor of VEGFR-2. EMMPRIN plays a central role in its activation not only in angiogenesis but also in increasing tumor cells malignant properties mediated by VEGFR-2. This should have implications in the design of new strategies to inhibit VEGFR-2 activation. Several innovative antiangiogenic drugs have recently been developed. Doxazosin, an hypertension drug was shown to decrease VEGFR-2/Akt/mTOR signalling and to exert antitumor effects in an animal model. Beside such monotherapy approach, a combinatory strategy using, for example, a dual EGFR inhibition together with anti VEGF treatment have recently shown an improved clinical benefit. In this context, our results propose a novel antiangiogenic approach using an inhibitor of EMMPRIN-VEGFR-2 interaction, which would be expected to be specific for tumor angiogenesis, as EMMPRIN is known to be highly expressed in cancer tissues. Its use in combination with an anti-angiogenic drug may have a greater impact on inhibiting angiogenesis and malignancy.

### Example 2

Design of Peptides for Blocking EMMPRIN/VEGFR-2 Interaction:

Based on inventors' results, several peptides were designed against EMMPRIN/VEGFR-2 binding site.

a) Peptide 1: 190-202 in SEQ ID NO:1 (log P around −2,7)

b) Peptide 2: 179-192 in SEQ ID NO:1 (log P around −4.5) Here the Ala replaces the Cys of the original sequence.

c) Peptide 3: 181-192 in SEQ ID NO:1 (log P around −3.7).

Effects of EMMPRIN Peptide Inhibitor (PI) on the Proliferation of HMEC Endothelial Cells (EC)

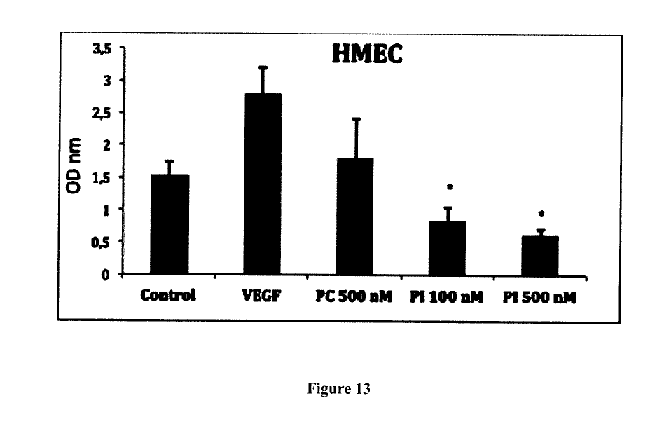

As VEGFR-2 has been implicated in regulation of endothelial cells migration/invasion and proliferation processes, we first investigated the effect of EMMPRIN inhibitor peptide (PI) on endothelial cell proliferation. MTT assay was used to measure the cell viabilities of HMEC cells peptide inhibitor (PI) or peptide control (PC) after treatment for 48 h. The results show that EMMPRIN PI but not CP significantly inhibited proliferation of HMEC cells (approx. 50%) (FIG. 13).

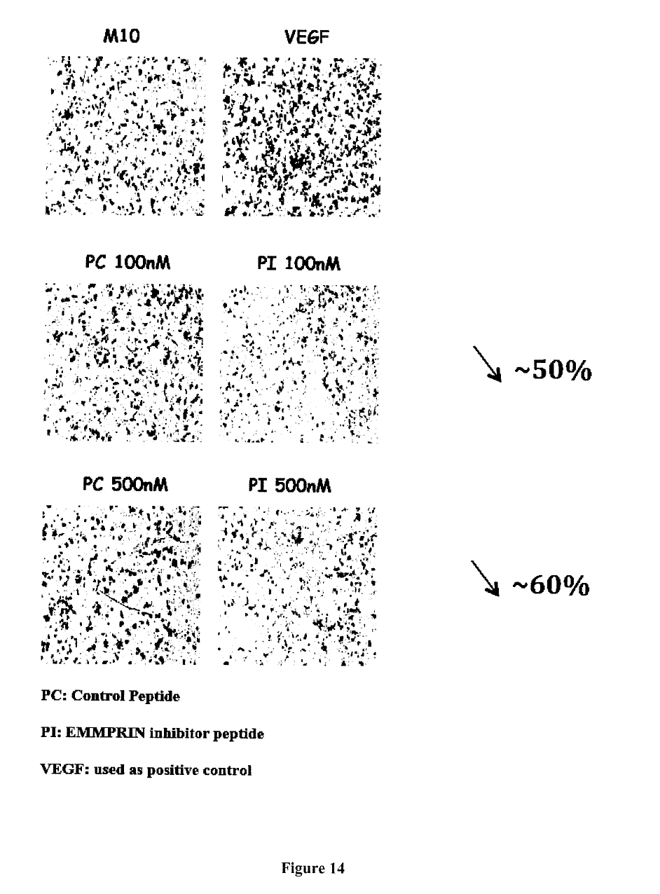

We next investigated the effect of EMMPRIN inhibitor peptide (PI) on invasive capacity of tumor cells. Using a modified Boyden chamber assay, PI decreased in M10 cell invasion capacity, showing a mean 60% decrease (500 nM) compared to control PC-treated cells (FIG. 14).

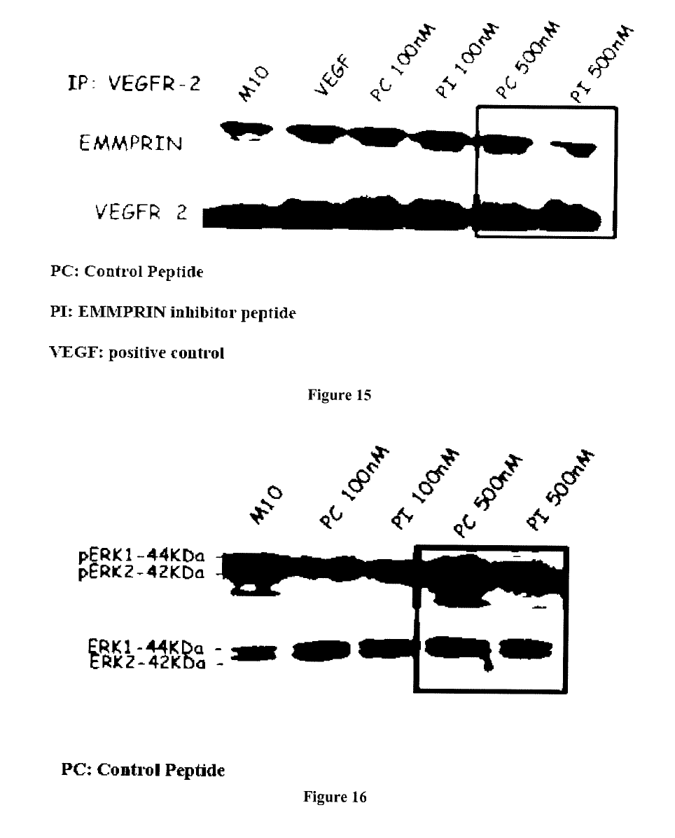

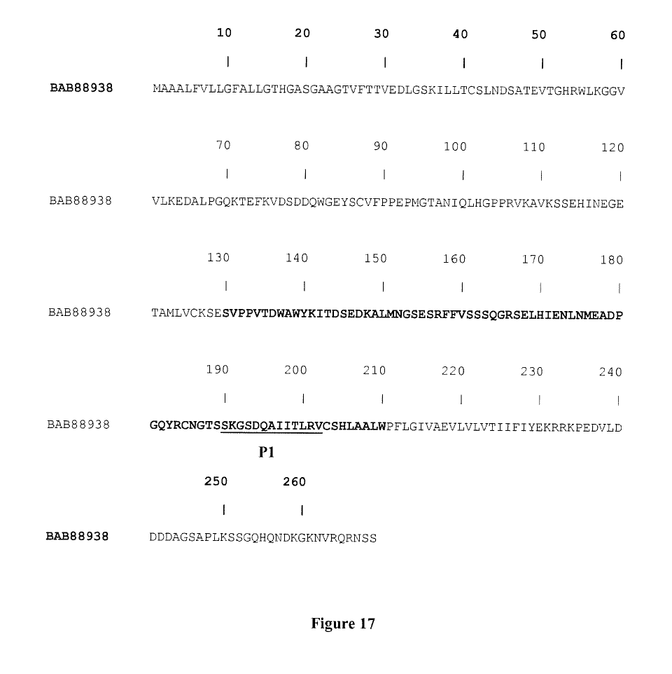

Immunoprecipitation experiments have shown that PI, at 100 nM and 500 nM, was able to decrease EMMPRIN/VEGFR-2 interaction. PI treated cells have also shown a significant decrease in ERK phosphorylation, known to be implicated in cell proliferation and invasion. No effect on ERK phosphorylation could be observed when the cells were treated with PC (FIGS. 15 and 16).

### REFERENCES

Throughout this application, various references describe the state of the art to which this invention pertains. The disclosures of these references are hereby incorporated by reference into the present disclosure.

1. Ferrara N, Gerber H P, LeCouter J: The biology of VEGF and its receptors. Nat Med 2003; 9(6):669-676.

2. Hicklin D J, Ellis L M: Role of the vascular endothelial growth factor pathway in tumor growth and angiogenesis. Journal of clinical oncology : official journal of the American Society of Clinical Oncology 2005; 23(5):1011-1027.

3. Shinkai A, Ito M, Anazawa H, Yamaguchi S, shikara K, Shibuya M: Mapping of the sites involved in ligand association and dissociation at the extracellular domain of the kinase insert domain-containing receptor for vascular endothelial growth factor. The Journal of biological chemistry 1998; 273(47):31283-31288.

4. Hyde C A, Giese A, Stuttfeld E, Abram Saliba J, Villemagne D, Schleier T, Binz H K, Ballmer-Hofer K: Targeting extracellular domains D4 and D7 of vascular endothelial growth factor receptor 2 reveals allosteric receptor regulatory sites. Molecular and cellular biology 2012; 32(19):3802-3813.

5. Barleon B, Totzke F, Herzog C, Blanke S, Kremmer E, Siemeister G, Marme D, Martiny-Baron G: Mapping of the sites for ligand binding and receptor dimerization at the extracellular domain of the vascular endothelial growth factor receptor FLT-1. The Journal of biological chemistry 1997; 272(16):10382-10388.

6. Voigt H, Vetter-Kauczok C S, Schrama D, Hofmann UB, Becker J C, Houben R: CD147 impacts angiogenesis and metastasis formation. Cancer Invest 2009; 27(3):329-333.

7. Tang Y, Nakada M T, Kesavan P, McCabe F, Millar H, Rafferty P, Bugelski P, Yan L: Extracellular matrix metalloproteinase inducer stimulates tumor angiogenesis by elevating vascular endothelial cell growth factor and matrix metalloproteinases. Cancer research 2005; 65(8):3193-3199.

8. Bougatef F, Quemener C, Kellouche S, Naimi B, Podgorniak M P, Millot G, Gabison E E, Calvo F, Dosquet C, Lebbe C et al: EMMPRIN promotes angiogenesis through hypoxia-inducible factor-2alpha-mediated regulation of soluble VEGF iso forms and their receptor VEGFR-2. Blood 2009; 114(27):5547-5556.

9. Bougatef F, Menashi S, Khayati F, Naimi B, Porcher R, Podgorniak M P, Millot G, Janin A, Calvo F, Lebbe C et al: EMMPRIN promotes melanoma cells malignant properties through a HIF-2alpha mediated up-regulation of VEGF-receptor-2. PloS one 2010; 5(8):e12265.

10. Quemener C, Gabison E E, Naimi B, Lescaille G, Bougatef F, Podgorniak M P, Labarchede G, Lebbe C, Calvo F, Menashi S et al: Extracellular matrix metalloproteinase inducer up-regulates the urokinase-type plasminogen activator system promoting tumor cell invasion. Cancer research 2007; 67(1):9-15.

11. Huet E, Gabison E E, Mourah S, Menashi S: Role of emmprin/CD147 in tissue remodeling. Connect Tissue Res 2008; 49(3):175-179.

12. Nabeshima K, Iwasaki H, Koga K, Hojo H, Suzumiya J, Kikuchi M: Emmprin (basigin/CD147): matrix metalloproteinase modulator and multifunctional cell recognition molecule that plays a critical role in cancer progression. Pathol Int 2006; 56(7):359-367.

13. Yan L, Zucker S, Toole B P: Roles of the multifunctional glycoprotein, emmprin (basigin; CD147), in tumour progression. Thromb Haemost 2005; 93(2):199-204.

14. Weidle U H, Scheuer W, Eggle D, Klostermann S, Stockinger H: Cancer-related issues of CD147. Cancer genomics & proteomics 2010; 7(3):157-169.

15. Muramatsu T, Miyauchi T: Basigin (CD147): a multifunctional transmembrane protein involved in reproduction, neural function, inflammation and tumor invasion. Histol Histopathol 2003; 18(3):981-987.

16. Sun J, Hemler M E: Regulation of MMP-1 and MMP-2 production through CD147/extracellular matrix metalloproteinase inducer interactions. Cancer research 2001; 61(5):2276-2281.

17. Koga K, Aoki M, Sameshima T, Hamasaki M, Egawa N, Seiki M, Toole B P, Suzumiya J, Nabeshima K: Synthetic emmprin peptides inhibit tumor cell-fibroblast interaction-stimulated upregulation of MMP-2 and tumor cell invasion. International journal of oncology 2011; 39(3):657-664.

18. Li Y, Wu J, Song F, Tang J, Wang S J, Yu X L, Chen Z N, Jiang J L: Extracellular membrane-proximal domain of HAb18G/CD147 binds to metal ion-dependent adhesion site (MIDAS) motif of integrin betal to modulate malignant properties of hepatoma cells. The Journal of biological chemistry 2012; 287(7):4759-4772.

19. Tang W, Hemler M E: Caveolin-1 regulates matrix metalloproteinases-1 induction and CD147/EMMPRIN cell surface clustering. The Journal of biological chemistry 2004; 279(12):11112-11118.

20. Sato M, Nakai Y, Nakata W, Yoshida T, Hatano K, Kawashima A, Fujita K, Uemura M, Takayama H, Nonomura N: EMMPRIN promotes angiogenesis, proliferation, invasion and resistance to sunitinib in renal cell carcinoma, and its level predicts patient outcome. PloS one; 8(9):e74313.

21. Yu XL, Hu T, Du J M, Ding J P, Yang X M, Zhang J, Yang B, Shen X, Zhang Z, Zhong W D et al: Crystal structure of HAb18G/CD147: implications for immunoglobulin superfamily homophilic adhesion. The Journal of biological chemistry 2008; 283(26):18056-18065.

22. Bahi A, Boyer F, Gumy C, Kafri T, Dreyer J L: In vivo gene delivery of urokinase-type plasminogen activator with regulatable lentivirus induces behavioural changes in chronic cocaine administration. Eur J Neurosci 2004; 20(12):3473-3488.

23. Naldini L, Blomer U, Gallay P, Ory D, Mulligan R, Gage F H, Verma I M, Trono D: In vivo gene delivery and stable transduction of nondividing cells by a lentiviral vector. Science 1996; 272(5259):263-267.

24. Shields J D, Emmett M S, Dunn D B, Joory K D, Sage L M, Rigby H, Mortimer P S, Orlando A, Levick J R, Bates D O: Chemokine-mediated migration of melanoma cells towards lymphatics--a mechanism contributing to metastasis. Oncogene 2007; 26(21):2997-3005.

25. Sali A, Blundell T L: Comparative protein modelling by satisfaction of spatial restraints. J Mol Biol 1993; 234(3):779-815.

26. Shi J, Blundell T L, Mizuguchi K: FUGUE: sequence-structure homology recognition using environment-specific substitution tables and structure-dependent gap penalties. J Mol Biol 2001; 310(1):243-257.

27. Katchalski-Katzir E, Shariv I, Eisenstein M, Friesem A A, Aflalo C, Vakser I A: Molecular surface recognition: determination of geometric fit between proteins and their ligands by correlation techniques. Proc Natl Acad Sci USA 1992; 89(6):2195-2199.

28. Chen R, Li L, Weng Z: ZDOCK: an initial-stage protein-docking algorithm. Proteins 2003; 52(1):80-87.

29. Cheng T M, Blundell T L, Fernandez-Recio J: pyDock: electrostatics and desolvation for effective scoring of rigid-body protein-protein docking. Proteins 2007; 68(2):503-515.

30. Lopez-Blanco J R, Garzon J I, Chacon P: iMod: multipurpose normal mode analysis in internal coordinates. Bioinformatics 2011; 27(20):2843-2850.

1. A polypeptide which comprises or consists of a sequence of at least 5 consecutive amino acids in SEQ ID NO:1 and which comprises at least one amino acid selected from the group consisting of Q182, R184, Q195, and T199.

2. The polypeptide of claim 1 which comprises or consists of a sequence of at least 5 consecutive amino acids in SEQ ID NO:1 and which comprises at least two amino acids selected from the group consisting of Q182, R184, Q195, and T199.

3. The polypeptide of claim 1 which comprises or consists of a sequence of at least 5 consecutive amino acids in SEQ ID NO:1 and which comprises at least three amino acids selected from the group consisting of Q182, R184, Q195, and T199.

4. The polypeptide of claim 1 which comprises or consists of a sequence of at least 5 consecutive amino acids in SEQ ID NO:1 and which comprises all of the amino acids Q182, R184, Q195, and T199.

5. The polypeptide of claim 1 which comprises or consists of a sequence of at least 5 consecutive amino acids in a region ranging from a residue at position 130 to an amino acid residue at position 210.

6. The polypeptide of claim 1 which comprises or consists of a sequence of at least 5 consecutive amino acids in the region ranging from a residue at position 190 to an amino acid residue at position 202.

7. The polypeptide of claim 1 which comprises 5; 6; 7; 8; 9; 10; 11; 12; 13; 14; 15; 16; 17; 18; 19; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; or 50 consecutive amino acids in SEQ ID NO:1.

8. The polypeptide of claim 1 which comprises or consists of a sequence having at least 70% of identity with a sequence which ranges from an amino acid residue at position 190 to a amino acid residue at position 202 in SEQ ID NO:1

9. The polypeptide of claim 1 which comprises or consists of a sequence having at least 70% identity with a sequence which ranges from an amino acid residue at position 179 to an amino acid residue at position 192 in SEQ ID NO:1.

10. The polypeptide of claim 1 which comprises or consists of a sequence having at least 70% of identity with a sequence which ranges from an amino acid residue at position 181 to an amino acid residue at position 192 in SEQ ID NO:1.

11. The polypeptide of claim 1 which is fused to heterologous polypeptide.

12. A nucleic acid molecule encoding for the polypeptide of claim 1.

13. A vector which comprises the nucleic acid molecule of claim 12.

14. A host cell transformed with the nucleic acid molecule of claim 12.

15. An antibody or aptamer which specifically binds to the polypeptide of claim 1.

16. (canceled)

17. A method of treating angiogenesis-related diseases or lymphoangiogenesis-related diseases in a subject in need thereof, comprising the step of administering to said subject the polypeptide of claim 1.

18. A method of treating angiogenesis-related diseases or lymphoangiogenesis-related diseases in a subject in need thereof, comprising the step of administering to said subject the nucleic acid of claim 12.

19. A method of treating angiogenesis-related diseases or lymphoangiogenesis-related diseases in a subject in need thereof, comprising the step of administering to said subject the aptamer of claim 15.

20. A method of treating angiogenesis-related diseases or lymphoangiogenesis-related diseases in a subject in need thereof, comprising the step of administering to said subject the antibody of claim 15.
